(19) United States
(12) Patent Application Publication
(10) Pub. No.: US 2010/0088773 A1
(43) Pub. Date: Apr. 8, 2010

(54) MUTATIONS IN LAMA2 GENE OF ZEBRAFISH

(75) Inventors: Thomas Edward Hall, Victoria (AU); Robert James Bryson-Richardson, Victoria (AU); Peter David Currie, Victoria (AU)

Correspondence Address: BARNES & THORNBURG LLP, P.O. BOX 2786, CHICAGO, IL, 60690-2786, US

(73) Assignee: VICTOR CHANG CARIDAC RESEARCH INSTITUTE LIMITED, Darlinghurst (AU)

(21) Appl. No.: 12/527,130

(22) PCT Filed: Feb. 15, 2008
(86) PCT No.: PCT/AU08/00210

§ 371 Date: Dec. 16, 2009

(30) Foreign Application Priority Data
Feb. 16, 2007 (AU) 2007900843
Sep. 3, 2007 (AU) 2007904764

(51) Int. Cl. A61K 49/00; C07H 21/04; C07K 14/00; C12Q 1/02; G01N 33/53; C12Q 1/68

(52) U.S. Cl. 800/3; 536/23.1; 530/350; 435/29; 435/7.1; 435/6

(57) ABSTRACT

The present invention relates to an isolated nucleic acid molecules encoding mutant lama2 gene of zebrafish, mutant zebrafish having mutations in the lama2 gene, fish models containing mutant zebrafish, and uses of the fish models.

### TECHNICAL FIELD

The present invention relates to mutations in Zebrafish genes that are useful in fish models for human disease.

### BACKGROUND ART

Congenital muscular dystrophies (CMDs) are a group of neuromuscular disorders with severe muscle hypotonia at birth or within the first months of life, generalised muscle weakness, contractures of variable severity and delayed motor milestones. The incidence of CMDs has been estimated to be approximately 1 in every 21,500 live births, with laminin alpha2 (lama2) deficient CMD (MDC1A) accounting for about 40-50% of the CMD cases in European countries. MDC1A is caused by genetic lesions in the lama2 gene. The classical phenotype is associated with complete LAMA2 deficiency, and pathological symptoms of muscle tissue degeneration, fibrosis and white matter abnormalities within the CNS.

Laminins are major structural components of basal laminae, and exist as heterotrimeric complexes of one alpha, one beta and one gamma chain. Particular complexes exhibit particular tissue specificities. There are currently 15 described mammalian complexes made up of varying combinations of 5 alpha chains, 3 beta chains, and 3 gamma chains. In addition, to their structural role, laminins also act as signalling molecules through receptors such as integrins and α-dystroglycan. The most studied complex to date has been laminin1, largely for historical reasons, since it was the first to be identified. Laminin1 consists of the laminin α1, β1 and γ1 chains. It appears early in epithelial morphogenesis in most embryonic tissues and is a major component of extra-cellular matrix. In the zebrafish, the α1, β1 and γ1 chains that make up the laminin1 complex are essential for normal embryonic development and have been shown to be particularly important in notochord morphogenesis and maintenance. The α2 subunit is known to be present in three complexes; laminins 211 and 221, expressed in the basal laminae of muscle fibres and the Schwann cells surrounding the peripheral nerves; and laminin 213, a little studied complex which is potentially the first non-basement membrane laminin.

Different hypotheses have been developed as to why lama2 deficiency leads to the onset of CMD. Most commonly, the proposed mechanism of cellular pathology centres upon the structural role of lama2, through its interaction with the dystrophin-associated glycoprotein complex (DGC), necessary for maintenance of sarcolemmal integrity. Laminin is known to bind directly to α-dystroglycan, the component of the DGC most distal to the sarcolemma, and thereby anchor the muscle cell membrane to the extracellular matrix. The notion of a structural link from the extracellular matrix through to the actin cytoskeleton being provided by the DGC is strengthened by the observation that a number of degenerative diseases of the skeletal muscle including DMD and certain limb girdle muscular dystrophies, are associated with abnormalities in components of the DGC. Traditionally, the accepted dogma regarding the cellular pathology of these diseases has been that loss of the structural link between the internal actin cytoskeleton and the cell membrane renders the sarcolemma vulnerable to mechanical damage, which, in turn, leads to fibre apoptosis and/or necrosis. However, in recent years mechanistic explanations of dystrophic pathologies have been challenged by hypotheses suggesting that signalling dysfunction could be more important than loss of sarcolemmal integrity. For instance, dystrophin, in addition to its structural role, serves as a scaffold for the assembly of a multi-component signal transduction complex, members of which also form integral parts of the DGC. In the case of lama2, both mechanistic and dysfunctional signalling explanations have been mooted for the pathology of MDC1A since, in vitro at least, laminin-integrin binding is involved in the regulation of myoblast proliferation and fusion. However, the laminin 211 complex is not strictly a DGC member, and is found external to the sarcolemma, forming a link between the DGC and the extracellular matrix. As such, the relative importance of loss of membrane integrity and/or signalling function in the pathology of the disease are unknown. In addition, lama2 deficient CMD is not normally associated with loss of DGC components or of Dystrophin itself. Also unlike DMD, MDC1A involves peripheral nerve defects, leading to a third hypothesis; that impaired neural function results in innervation abnormalities and/or relatively little electrical stimulation and contraction of myofibres, effectively causing denervation atrophy. Thus the actual basis of the pathology evident in lama2 deficient CMD remains to be determined.

Zebrafish provide a number of unique opportunities over existing vertebrate models for skeletal muscle research, due to their optical transparency and ex utero development, which allows direct observation of developmental processes. At the same time it is possible to carry out sophisticated embryological and genetic manipulations within the intact embryo. Zebrafish are also highly fecund and amenable to projects necessitating large-scale familial rearing schemes, such as random mutagenesis. Furthermore, the myotomal muscle of the zebrafish axis represents a highly manipulable paradigm where presumptive muscle cells can undergo specification, proliferation and fusion, followed by fibre differentiation and attachment within a matter of hours.

Numerous zebrafish muscle mutants have been isolated from large-scale screening programs designed to generate mutations in genes essential for the formation and maintenance of individual tissue and organ systems. Of particular interest amongst these is a class of mutations whose phenotypes bear superficial resemblance to mammalian dystrophic models. Animals homozygous for mutations within these genes display skeletal muscle specific degeneration. Previously, the applicants reported that one member of the dystrophic class, sapje (sap), possesses nonsense mutations within the zebrafish homologue of the Dystrophin gene, causative for Duchenne's muscular dystrophy (DMD) in humans (Bassett, D. I., R. J. Bryson-Richardson, et al. (2003). “Dystrophin is required for the formation of stable muscle attachments in the zebrafish embryo.” Development 130(23): 5851-60). A detailed examination of the sap phenotype revealed that degeneration results from the failure of muscle cell attachments at the end of muscle fibres, in a manner consistent with a structural failure of the dystrophin linkage on the intracellular side of the membrane. This novel pathological process at the site of the embryonic myotendinous junction has hitherto been overlooked in the traditional dystrophic animal models.

The present inventors have obtained novel null alleles of lama2 in the zebrafish, in which muscle pathology can be directly observed in real time using time lapse photomicroscopy.

### DISCLOSURE OF INVENTION

In a first aspect, the present invention provides isolated zebrafish genetic strain having a laminin mutant phenotype resulting from a mutation within the zebrafish lama2 gene.

Preferably, the mutant has a candyfloss phenotype as defined herein.

Preferred mutants are termed candyfloss (caf^{teg15a}, caf^{tk209}), being the currently identified mutant alleles within the zebrafish lama2 gene, and any combinations or other alleles that are generated being defined as mutations within the zebrafish lama2 gene or any zebrafish strain resulting from a mutation within the zebrafish lama2 gene.

As the present inventors have developed zebrafish genetic strains having a lama2 mutant phenotype resulting from one or more mutations within the zebrafish lama2 gene, it will be appreciated that other mutations of the zebrafish lama2 gene would be contemplated from the teaching of the present invention.

It will be appreciated that further mutants, progeny, fry, eggs, gametes are also included in the scope of the present invention.

In a second aspect, the present invention provides a fish model of mammalian congenital muscular dystrophy comprising an isolated zebrafish according to the first aspect of the present invention.

Preferably, the model is of human muscular dystrophy.

In a third aspect, the present invention provides a method for screening agents having potential activity on muscular dystrophy comprising:

(a) providing a fish model according to the second aspect of the present invention;
(b) exposing the zebrafish to an agent; and
(c) determining any affect of the agent on a genetic or physical characteristic of the zebrafish or its progeny.

The agent may be a drug candidate, chemical, nucleic acid and the like.

The agent may be administered by direct dilution in raising media, or direct administration to the fish by any suitable means.

The effect may be determined by any visual or light microscopic technique including those that utilise transgenic reporter gene expression to monitor muscle integrity. They include, but not limited to, simple optical inspection of living muscle tissue, birefringency of muscle tissue using polarised light, the use of fluorescent protein transgenic lines driven by muscle specific promoter(s), the use of immunohistochemistry, using antibodies directed against muscle specific epitopes and in situ hybridisation for muscle specific gene expression.

In a fourth aspect, the present invention provides an agent determined to have activity on muscular dystrophy by the method according to the third aspect of the present invention.

In a fifth aspect, the present invention provides a method for monitoring or testing the effect of an agent having activity on muscular dystrophy comprising:

(a) providing a fish model according to the second aspect of the present invention;
(b) exposing the zebrafish to the agent; and
(c) monitoring the effect of the agent on a genetic or physical characteristic of the zebrafish or its progeny.

In a sixth aspect, the present invention provides an isolated nucleic acid molecule encoding lama2 gene, nucleic acid molecules complementary to the nucleic acid molecule encoding the lama2 gene, nucleic acid molecules that hybridise, preferably under stringent conditions, to the nucleic acid molecule encoding lama2 gene. Preferably, the cDNA sequence is set out in FIG. 10 (SEQ ID NO: 1).

In a seventh aspect, the present invention provides an isolated lama2 protein. Preferably; the protein has the amino acid sequence set out in FIG. 11 (SEQ ID NO: 2).

In a eight aspect, the present invention provides an isolated nucleic acid molecule encoding a candyfloss phenotype as defined herein. Preferably, the mutations are candyfloss (caf^{teg15a}, cat^{tk209}) as set out in FIG. 12 (SEQ ID NO: 3) and FIG. 13 (SEQ ID NO: 4).

The present inventors have identified a class of zebrafish mutations as candidates for mutations in human muscular dystrophy disease genes. The molecular lesion in one of these mutations, candyfloss has been identified. The candyfloss phenotype resulted from mutations within the lama2 gene, human mutations in which result in Laminin alpha 2-deficient congenital muscular dystrophy (MDC1A) the most common form of congenital muscular dystrophy.

The present inventors have established a formal link between the phenotype of this particular class of zebrafish mutations and human muscular dystrophies. The phenotypes of these mutations have been characterised in detail, including that of the candyfloss mutations analysed in the results section below. These mutations exhibit muscle weakness in a similar manner to that described to occur in human patients. The phenotype of candyfloss (the zebrafish lama2 mutations) has been characterised in the most detail.

A number of attributes of zebrafish biology and development lend themselves to the implementation of a high through out screening rationales for genetic and pharmacological modifiers of the dystrophic phenotype. External fertilisation, high fecundity, optical transparency and small size of the embryos will allow us to directly screen for chemicals or second site mutations that modulate the dystrophic phenotype. These findings would form the basis of drug design for treatment of the human dystrophic condition.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred embodiments will be described with reference to the following drawings and examples.

### BRIEF DESCRIPTION OF THE DRAWINGS

FIG. 1 shows survival curve for homozygous caf embryos, and unaffected siblings. 80 embryos showing the caf phenotype were split into 4 replicates of 20 per pot. The majority of the mortality occurred between days 11-13 post-fertilization. Two homozygotes out of the original 80 survived to adulthood (3 months).

FIG. 2 shows fibre detachment in homozygous caf embryos. Panels show individual frames from a time-lapse movie taken under DIC, at 0.5 frames/second. A single fibre is seen detaching from the myoseptum and retracting into the somite.

FIG. 3 shows Evans blue dye (EBD) injections into the pre-cardiac sinus results in uptake by cells with compromised membranes. EBD is not taken up by cells in homozygous caf embryos at 72 hpf after fibre detachment (Ai, Aii, Bi, Bii), indicting that fibre detachment is not associated with loss of sarcolemmal integrity. Uptake of EBD is seen at 120 hpf in apoptotic cells which have taken on a “granular” appearance under DIC. Panels Ci, Cii, Di, Dii represent a positive control and show Evans blue uptake in sap (Dystrophin deficient) embryos. i) Red fluorescence channel, ii) DIC image.

FIG. 4 shows the genomic mapping strategy and numbers of recombinant embryos at each microsatellite marker.

FIG. 5 shows expression of lama2 mRNA at 72 hpf during zebrafish development. i) homozygous caf embryo shows little or no lama2 mRNA expression indicative of nonsense mediated decay. ii) Wildtype embryo shows myotomal expression of lama2 mRNA.

FIG. 6 shows that injection of antisense morpholino oligonucleotides against lama2 phenocopies the caf phenotype in 72 hpf embryos. A—Wildtype embryo, B—antisense morpholino injected embryo, C—teg15a homozygote.

FIG. 7 shows staining at 72 hpf with α-bungarotoxin, which marks the neuromuscular junctions (NMJs) and reveals that there is no difference in the extent of innervation between homozygous caf embryos and unaffected siblings. Differences in the pattern of innervation simply reflects retraction of NMJs with detached fibres in caf embryos.

FIG. 8 shows that fibre detachment occurs with the extracellular matrix rather than at the sarcolemma. Staining with antibodies for dystrophin, β-dystroglycan and laminin1 all show retraction of their epitopes into the somite with the ends of detaching fibres. This indicates that attachment failure occurs external to the sarcolemma and the dystrophin associated glycoprotein complex.

FIG. 9 shows transmission electron micrographs of the vertical myosepta in caf and unaffected sibling embryos at 72 hpf and 120 hpf. At 72 hpf the phenotype is subtle, but under high power (×7100-54000) tearing of the myosepta is apparent. In contrast, by 120 hpf, even under low power (×2400), the myosepta display advanced fibrosis and continued tearing. Under high power, portions of extracellular matrix can be seen to infiltrate the myotome, apparently pulled along with detaching fibres. The myosepta are greatly increased in diameter, and show condensed collaged fibres.

FIG. 10 shows cDNA sequence for the zebrafish wild-type lama2 mRNA (SEQ ID NO: 1).

FIG. 11 shows the deduced amino acid sequence for the zebrafish wild-type lama2 protein (SEQ ID NO: 2).

FIG. 12 shows cDNA sequence for the zebrafish teg15a lama2 sequence (SEQ ID NO: 3). Affected residue (G-T change) is underlined and flanked with asterisks.

FIG. 13 shows cDNA sequence for the zebrafish tk209 lama2 sequence (SEQ ID NO: 4). Affected residue (G-A change) is underlined and flanked with asterisks.

### MODE(S) FOR CARRYING OUT THE INVENTION

The present inventors have obtained two novel null alleles of lama2 in the zebrafish, in which muscle pathology can be directly observed in real time using time lapse photomicroscopy. Our analyses lead to a hypotheses of lama2 function that is likely to be clinically significant. We clearly show that in the zebrafish model of MDC1A, the primary mechanism of pathology is through fibre detachment induced by mechanical loading of the fibre. In contrast to models of DMD, this fibre detachment occurs in the absence of major sarcolemmal disruption or loss of components of the DGC. Using transmission electron microscopy, we demonstrated a loss of integrity of the extracellular matrix and subsequent fibrosis. In addition, we showed that early myoblast proliferation and fusion are unaffected, suggesting that in this model, loss of the signal transducing activity of lama2 does not lead to muscle pathology. Similarly, formation and function of the primary motor neurons was normal and no differences where found in innervation between homozygous caf embryos and unaffected siblings. Furthermore, fibre detachment was dependant upon motor activity, leading to the conclusion that peripheral nerve defects do not contribute to pathology in this system. The zebrafish caf model of MDC1A should prove invaluable in future studies of gene and cell based therapies, and in chemical and genetic modifier screens.

### Results

### Identification of the Candyfloss Locus

The dystrophic mutants (class A4; Granato, M., F. J. van Eeden, et al. (1996). “Genes controlling and mediating locomotion behavior of the zebrafish embryo and larva.” Development 123: 399-413) from the Tubingen screen were characterised by muscle degeneration shortly after formation. They have impaired motility in the early larval phase, and show reduced muscle birefringency under polarised light. Complementation was performed between mutants sapje ta222a, softy tm272a, and “unresolved” alleles tf212b, teg15a, tk209a. Of these, only teg15a and tk209 were found to be in the same complementation group and distinct from already defined loci. We named this novel dystrophic mutant candyfloss (caf^{teg15a}, caf^{tk209}) due to the severe nature of the progressive muscle loss and the shape of dystrophic muscle fibres evident in homozygous mutants. The gross phenotype of the two caf alleles was indistinguishable. Consequently all phenotypic analysis was performed on caf^{teg15a}.

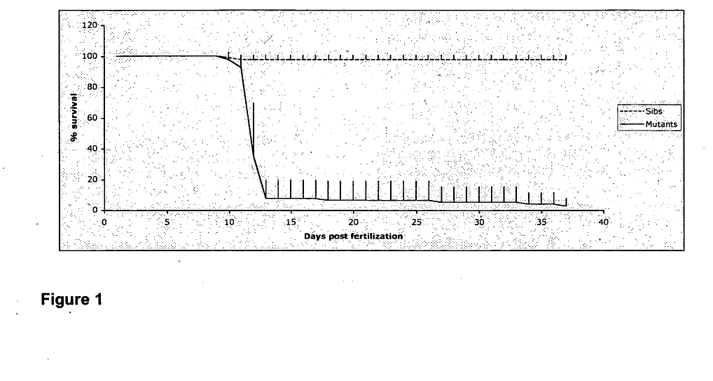

Initial formation of myotomal muscle is completely normal in caf embryos. However, immunohistochemistry using antibodies against slow and fast myosin heavy chains revealed that at 36 hpf (hours post fertilization), shortly after elongation and fusion of myofibres the first pathology becomes evident. The dystrophic appearance of the muscle is caused by detachment and retraction of muscle fibres from the vertical myosepta, which form the somite boundaries. Detachment first occurs in the slow muscle layer at the periphery of the myotome, which are the first fibres in the embryo to differentiate and function. This is closely followed by detachment in the deeper, fast muscle layer. In live embryos, the caf phenotype is first visible under DIC at 48 hours. At this time, the first myotomal lesions can be seen in a small proportion of embryos within a clutch. There is some variability in the severity of the phenotype between homozygotes, and the phenotype only becomes fully penetrant after 72 hpf, around the time of hatching. Mutant embryos often need to be manually dechorionated at this time since many are unable to extricate themselves and otherwise die within the chorion. Muscle damage does not affect all somites equally. Whereas a particular somite may appear normal, its neighbour might contain virtually no intact fibres. Such stochastic fibre damage is a hallmark of muscular dystrophy of human patients and mammalian animal models alike, as well as the dystrophin-deficient zebrafish mutation sapje. Muscle birefringence under polarised light is much reduced in affected somites. Mutants have severely affected motility, although this does not prevent the “swim up” behaviour necessary for inflation of the swim-bladder. The majority of mortality was found to occur suddenly around days 11-13. However, a small number of homozygote mutants survived this critical period and reached adulthood (2/80), although these individuals have not yet reproduced (FIG. 1).

### Muscle Fibre Detachment is Induced by Motor Activity

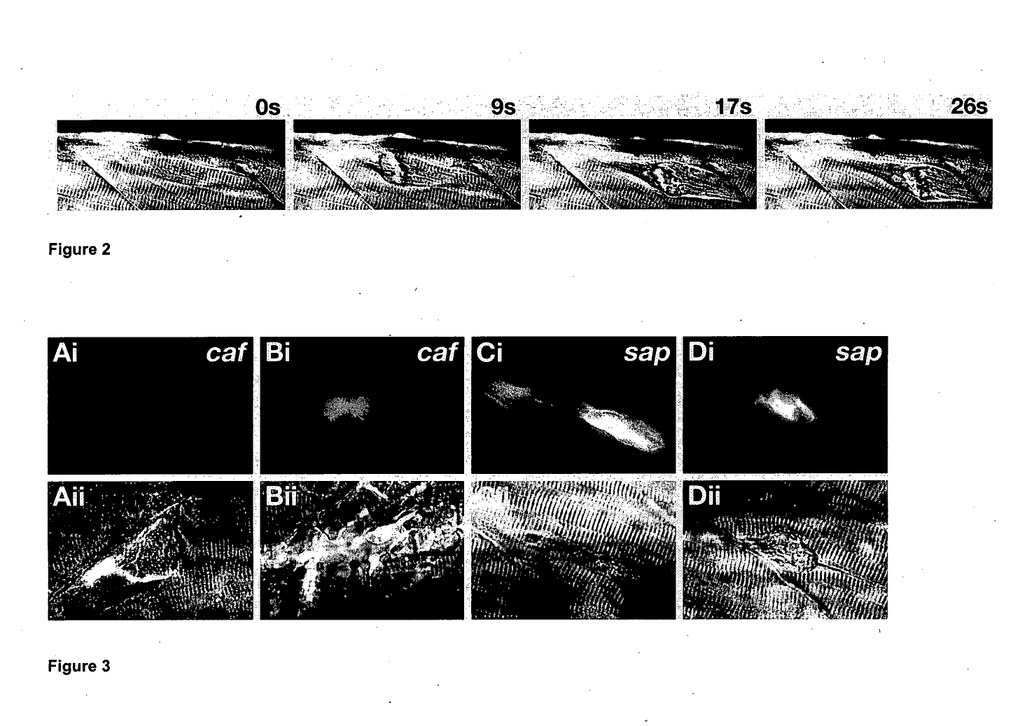

The stochastic pattern of muscle damage between somites led us to investigate whether muscle damage was related to motor activity. Raising embryos under anaesthetic resulted in complete suppression of the phenotype by 72 hpf (n=0/40, Table 1). Conversely, mechanically overloading the muscle of mutant larvae greatly increased both the severity and incidence of the fibre pathology within these animals (Table 1; Table 2). Mechanical loading of fibres was achieved by stimulating larvae to swim through raising media to which had been added the inert cellulose polymer, methyl-cellulose, which increased the effective viscosity of the surrounding media through which the larvae were required to swim. Raising embryos in 0.6% methyl-cellulose led to detached fibres in virtually every somite of caf homozygous mutants (n=11/11, Table 1) but had no effect in wildtype siblings (n=29/29). The nature of this fibre loss could be captured in real time via the use of an anaesthetic recovery protocol. Previously anaesthetized mutants were transferred into a highly viscous 3% methyl-cellulose solution, dissolved in raising media that contained no anaesthetic. Upon anaesthetic recovery, the partially immobilised muscle began to contract against the high viscosity of the mounting media inducing a very rapid fibre pathology. Fibre detachment induced under these conditions could be visualised using time-lapse photomicroscopy and this data is provided in (FIG. 2). It is clear from these analyses that the phenotype of homozygous caf mutants results from contraction-induced fibre detachment from the ends of the muscle fibres, and that the severity of this detachment phenotype is proportional to the load under which muscle fibres are placed.

TABLE 1

| | | | | | 0.6% Methyl- | |
|---|---|---|---|---|---|---|
| | Anaesthetized | | E3 only | | cellulose | |
| | Mutant | Sib | Mutant | Sib | Mutant | Sib |
| Genotype | 10 | 30 | 13 | 27 | 11 | 29 |
| Phenotype | 0 | 40 | 13 | 27 | 11 | 29 |

Penetrance of the caf phenotype in relation to mechanical loading of muscle fibres. Clutches of 40 embryos were raised in either anaesthetic, embryo media (E3) only, or 0.6% methyl-cellulose, between 48 hpf and 120 hpf. Embryos were subsequently genotyped by restriction analysis. Genotypically mutant embryos raised in anaesthetic did not display the caf phenotype.

TABLE 2

| | | 0.6% methyl |
|---|---|---|
| | E3 only | cellulose |
| Severity of | ++, +, +, +, ++, | +++, +++, +++, |
| phenotype | +, +, ++, +, +, | +++, +++, +++, |
| | ++, +, + | +++, +++, +++, |
| | | +++, +++ |

Severity of the caf phenotype in the homozygous mutants in Table 1, in relation to mechanical loading of muscle fibres. Embryos were scored as either mild (+), where only a small number of fibres in a few somites are affected, medium (++), or severe (+++), where a large number of fibres in virtually all somites are affected. Embryos raised in 0.6% methyl-cellulose showed a more severe phenotype than those raised in E3 alone.

Fibre Detachment is not Associated with Loss of Sarcolemmal Integrity.

The similarity of the caf phenotype to models of DMD led us to investigate whether sarcolemmal integrity was also compromised in these animals. Evans blue dye (EBD) is a small molecular weight dye which fluoresces in the red channel under UV light. Whilst the sarcolemma of physiologically normal cells is impermeable to EBD, it selectively accumulates in cells in which the sarcolemma has been torn. Injection of EBD into the precardiac sinus results in the passage of dye through the larval circulatory system, and consequent uptake by cells with compromised membranes. Unlike in sap fish, no uptake of EBD was seen in caf homozygotes at 72 hpf by retracted or non-retracted fibres. On the contrary, EBD fluorescence was seen to pool in the inter-fibre myotomal lesions created by fibre retraction (FIG. 3), indicating that sarcolemmal integrity was maintained. By 120 hpf, apoptotic/necrotic retracted fibres that had taken on a granular appearance under DIC, showed EBD infiltration.

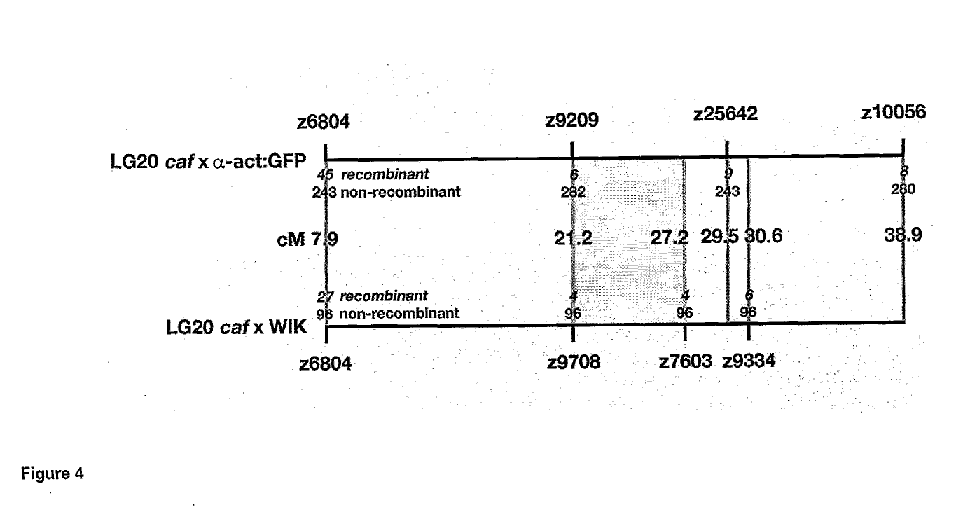

The caf^{teg15a} and caf^{tk209} Alleles Map to a Region Containing the Zebrafish Orthologue of Laminin Alpha2
A first-pass map position for caf^{teg15a} was established using standard bulked segregant analysis (Giesler 2002) to markers z6804 and z10056 on linkage group 20 with reference to the simple sequence repeat (SSR) map publicly available. Using a fine mapping strategy, this region was further refined to the flanking markers z9708 and z7603. The closest markers we were able to place on the Ensembl genome assembly were z10901 and z25642 which flanked a region of ˜0.89 mb, containing 23 transcripts (FIG. 4). In the centre of this region was a portion of the zebrafish orthologue of laminin alpha2 (lama2), which in humans is causative for LAMA2 deficient congenital muscular dystrophy. To confirm the genomic position of zebrafish lama2, radiation hybrid mapping was carried out on the LN54 panel (Hukriede, N. A., L. Joly, et al. (1999). “Radiation hybrid mapping of the zebrafish genome.” Proc Natl Acad Sci USA 96(17): 9745-50) using gene-specific primers to a portion of the lama2 open reading frame. Using this approach, significant linkage was found to marker z6804 (one, of those implicated in the initial bulked segregant analysis).
Lama2 mRNA Expression is Reduced in Mutant Embryos

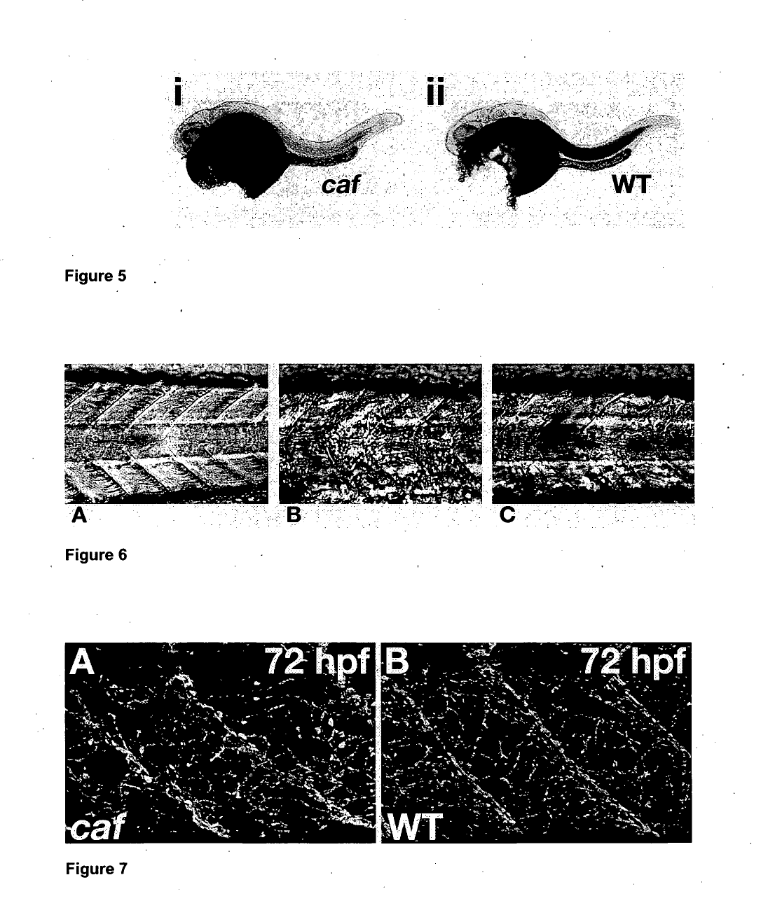

To investigate Lama2 as a candidate for causation of the caf phenotype, we carried out in situ hybridisation for the lama2 mRNA on caf mutant and sibling embryos (FIG. 5). Lama2 is expressed predominantly in the skeletal muscle during development. The transcript is first detected in an adaxial cell pattern, which are the first muscle cells to differentiate and express other myofibrillar markers such as MyHC. By 72 hpf, the transcript level is much reduced in the skeletal muscle and only a weak signal is detectable by in situ hybridisation. Unlike dystrophin, and a number of other muscle specific mRNAs, the lama2 message is not localised to the ends of the muscle fibres. lama2 expression was also seen in the fin muscles at day 5 pf. Furthermore, we noted that approximately 25% of embryos showed a weaker staining pattern for the lama2 expression than their siblings. At 72 hpf, the lower level of lama2 message correlated with embryos exhibiting the caf phenotype (n=13/42).

The caf^{teg15a} and caf^{tk209} Strains Contain Stop Mutations in the lama2 Open Reading Frame and are Phenocopied by Anti lama2 Morpholinos

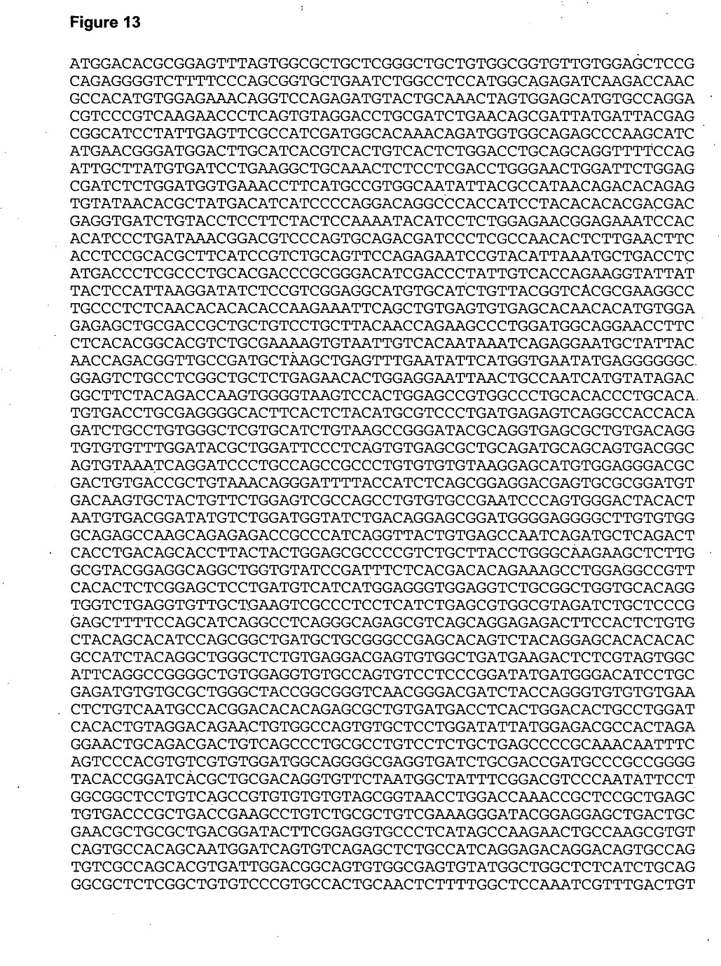

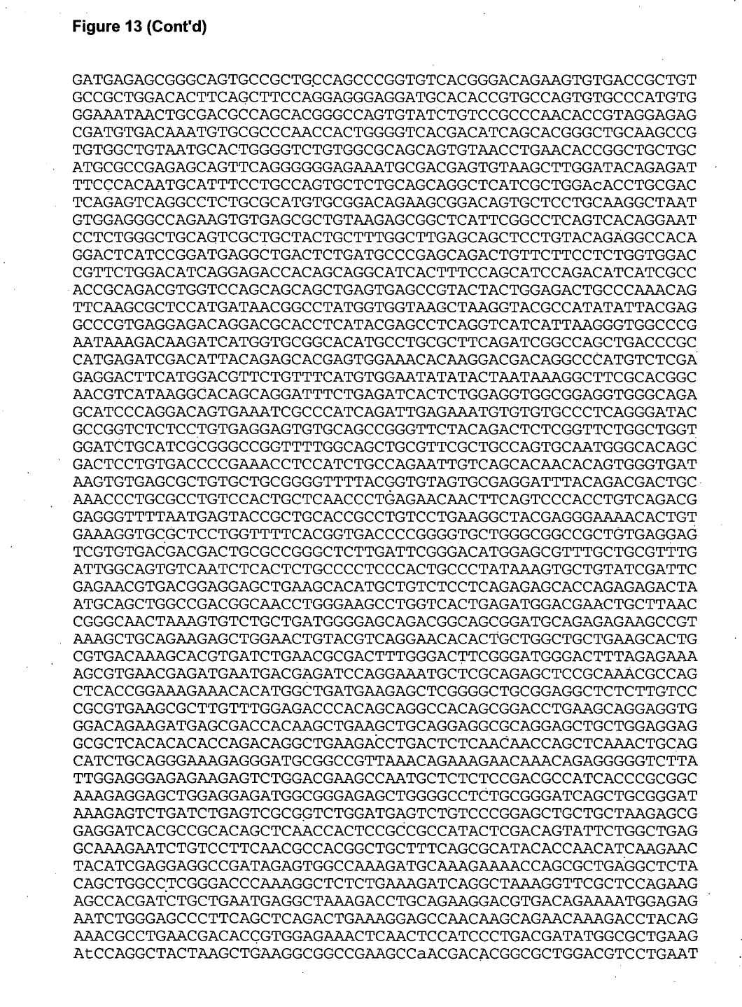

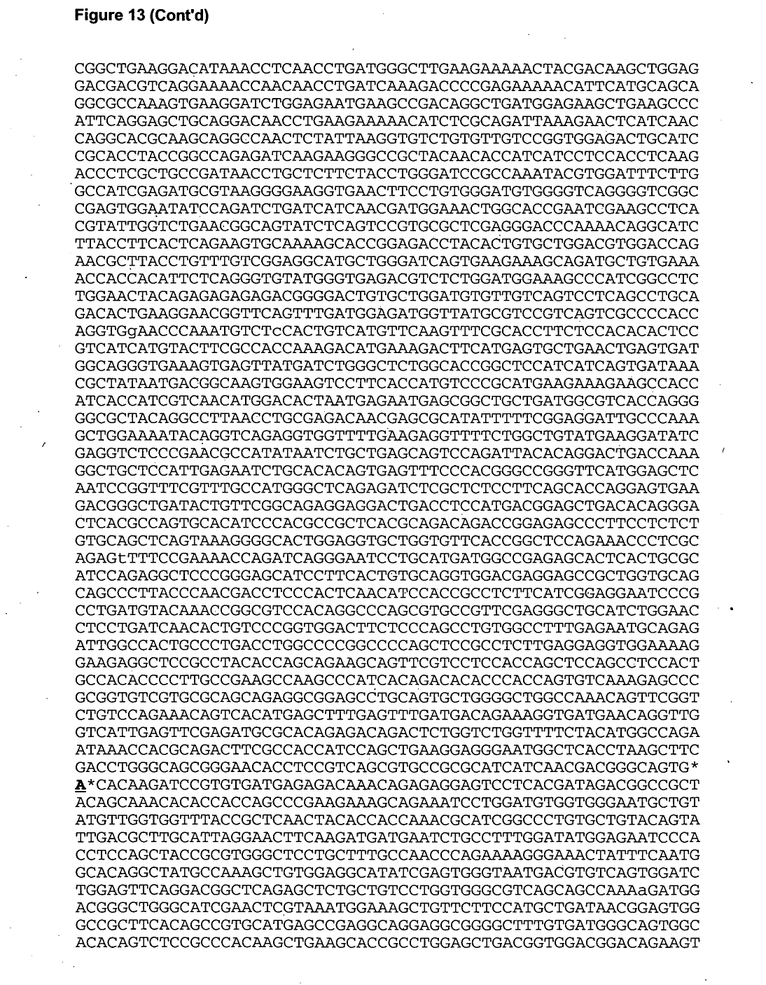

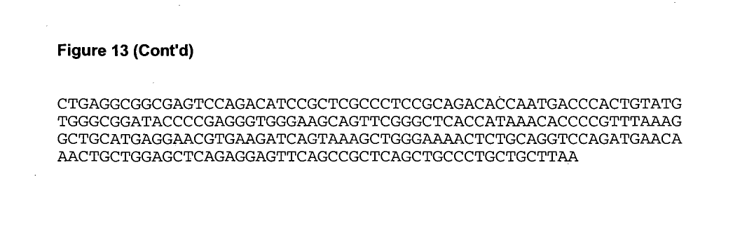

The high degree of mis-assembly in the genomic region necessitated bioinformatic interrogation of the interval and surrounding sequence using a hidden Markov model Eddy, S. R. (1998). “Profile hidden Markov models.” Bioinformatics 14(9): 755-63) of the mouse and human lama2 amino acid reference sequences. After identification of 55 putative coding exons, a contiguous genomic structure was predicted using Genewise Birney, E., M. Clamp, et al. (2004). “GeneWise and Genomewise.” Genome Res 14(5): 988-95). All putative exons were successfully sequenced using flanking primers within adjacent introns. Premature stop codons were found in the zebrafish homologue of human exon 60 in both caf^{teg15a} and caf^{tk209} (Table 3). Twenty-four affected and 24 unaffected progeny from each allele strain were genotyped by initial restriction analysis, followed by sequencing, demonstrating segregation of the mutations with the dystrophic phenotypes (FIG. 13).

TABLE 3
Results of genotype analysis that shows that the teg15a
and tk209 mutations segregate with the phenotype

| Mutant | | teg15a locus | | | tk209 locus | | |
|---|---|---|---|---|---|---|---|
| teg15a | Affected | | | 24 | 24 | | |
| | Unaffected | 9 | 15 | | 24 | | |
| tk209 | Affected | 24 | | | | | 24 |
| | Unaffected | 24 | | | 8 | 16 | |

To further demonstrate that mutations in the zebrafish lama2 gene cause a dystrophic phenotype, we injected antisense morpholino oligonucleotides into the first blastmere of wild-type embryos. MO-Lama2-1 was a translation blocking morpholino designed to cover the intiation codon, and MO-Lama2-60 was designed to overlap the boundary of the zebrafish homologues of human exons 59 and 60, inducing exon-skipping of exon 60, and a frameshift in exon 61, to result in a truncated protein.

Injection of either morpholino did not cause non-specific abnormalities at levels above sham-injected embryos, and phenocopied the caf phenotype (FIG. 6). Thus, we concluded that the mutations we have identified in lama2 cause the caf^{teg15a} and caf^{tk209} phenotypes respectively.

### Innervation

One model of the cellular pathology in lama2 deficiency is that innervation defects lead to denervation atrophy. To investigate this hypothesis in the context of the caf model we investigated innervation by the primary motor neurons. We used TRITC conjugated α-bungarotoxin, which binds irreversibly to the neuromuscular junction (NMJ), and fluoresces in the red channel. We investigated the innervation pattern in homozygous caf and sibling embryos. We detected no difference in the extent of innervation between homozygous caf and unaffected sibling embryos (FIG. 7). There was a noticeable difference in the pattern of innervation between affected and unaffected embryos. However, this appeared to simply reflect retraction of NMJs along with detached fibres.

Fibre Detachment Occurs on the Extracellular Side of the Membrane at the MTJ, Rather than at the Sarcolemma

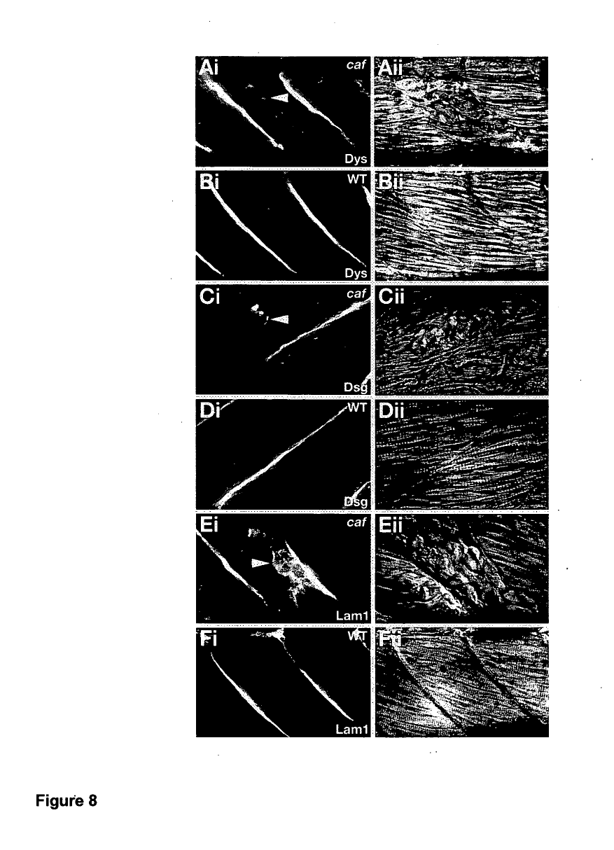

The maintenance of membrane integrity in retracted fibres led us to investigate the effects of the caf phenotype on DGC associated proteins at the sarcolemma. Dystrophin and β-dystroglycan (βDG) proteins are known to be expressed at the junctional sarcolemma after 36 hours (Bassett et al 2003). In addition, the laminin1 (α1, β1, γ1) complex is detectable within the extracellular matrix of the vertical myoseptum at this time. Dystrophin, βDG and Lam1 expression at the MTJ were unaffected in caf embryos. Furthermore, all epitopes showed a retraction with the detached fibre ends (FIG. 8), consistent with attachment failure within the ECM rather than at the sarcolemma.

### Lama2 Mutants Display Ultrastructural Defects at the Myotendinous Junction

The vertical myosepta, dividing the trunk somites are composed mainly of dense collagen, and their structure and function are similar to that of the mammalian tendon. As such, they are regarded as the homologous or analogous tissue. To investigate the detachment of fibres at the zebrafish MTJ, in the context of lama2 deficiency, we used transmission electron microscopy (TEM) on caf embryos. We compared mutant and wildtype sibling embryos at two separate period of development, firstly at 72 hpf, when the phenotype is fully penetrant but still relatively mild, and at 120 hpf when the phenotype is more severe.

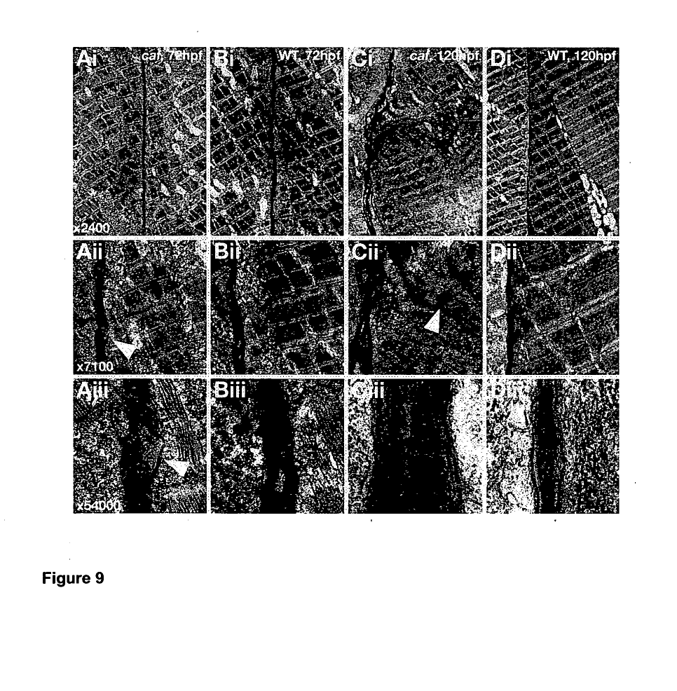

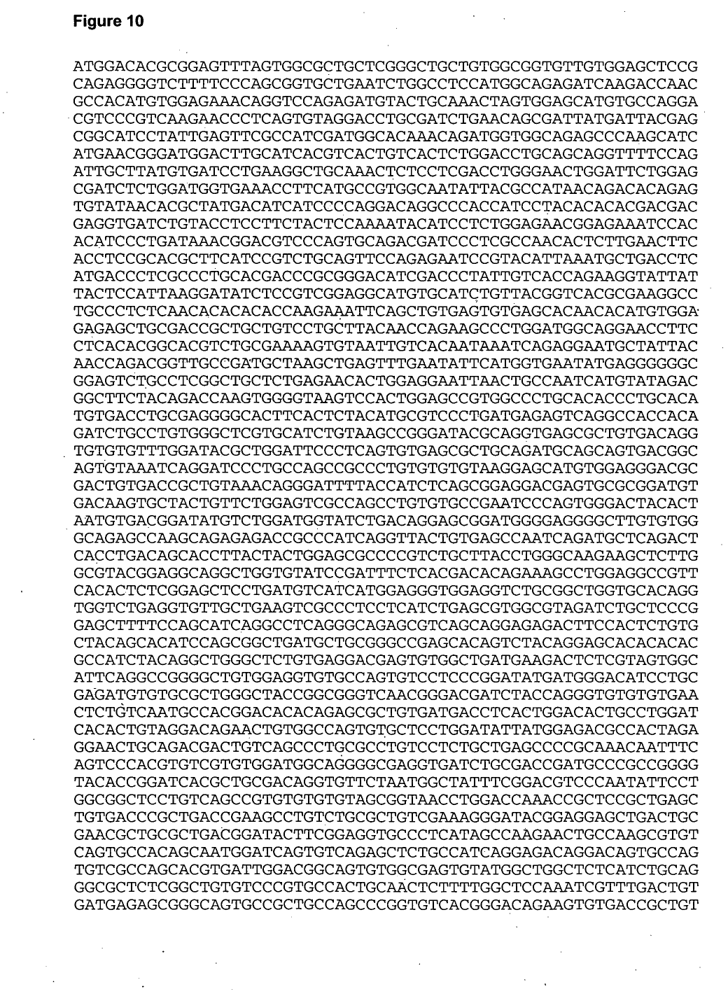

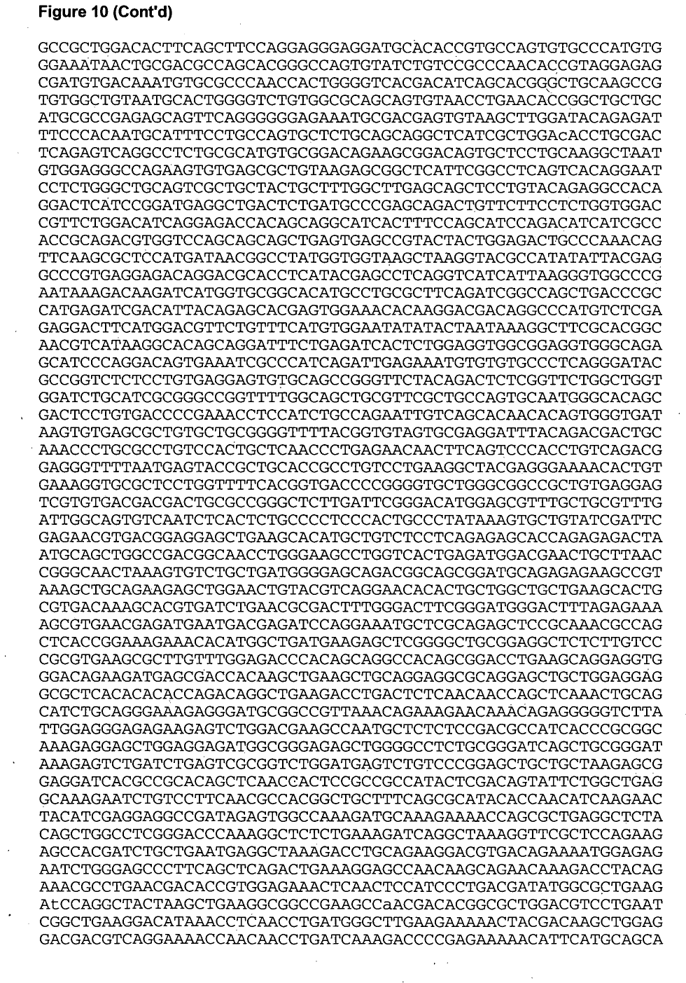

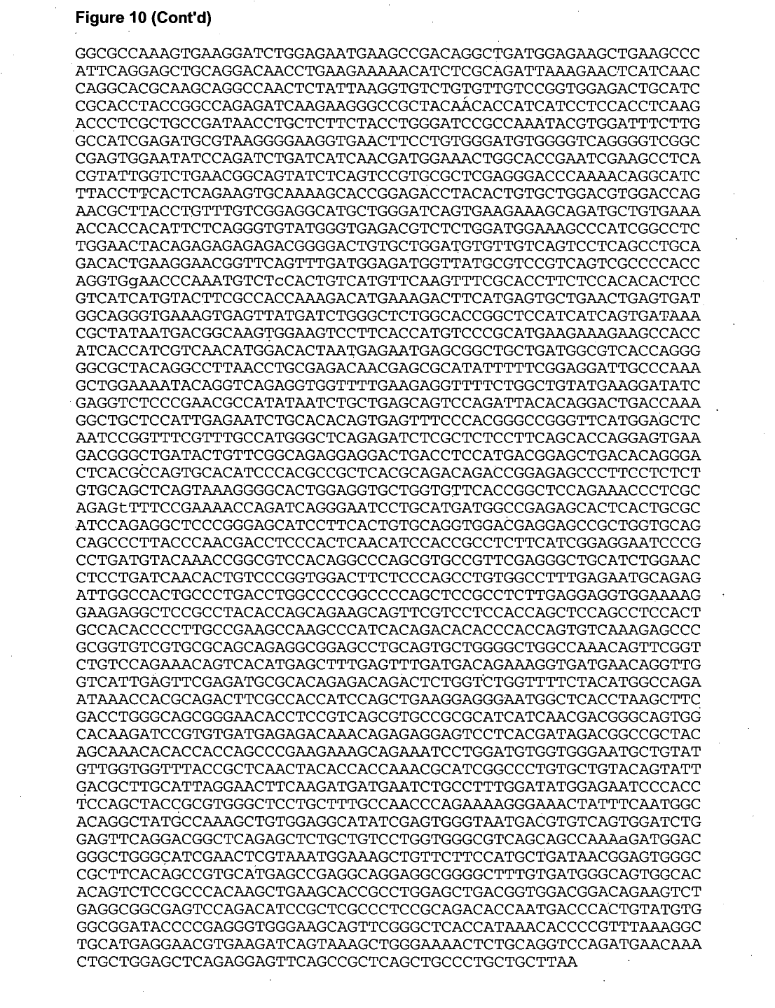

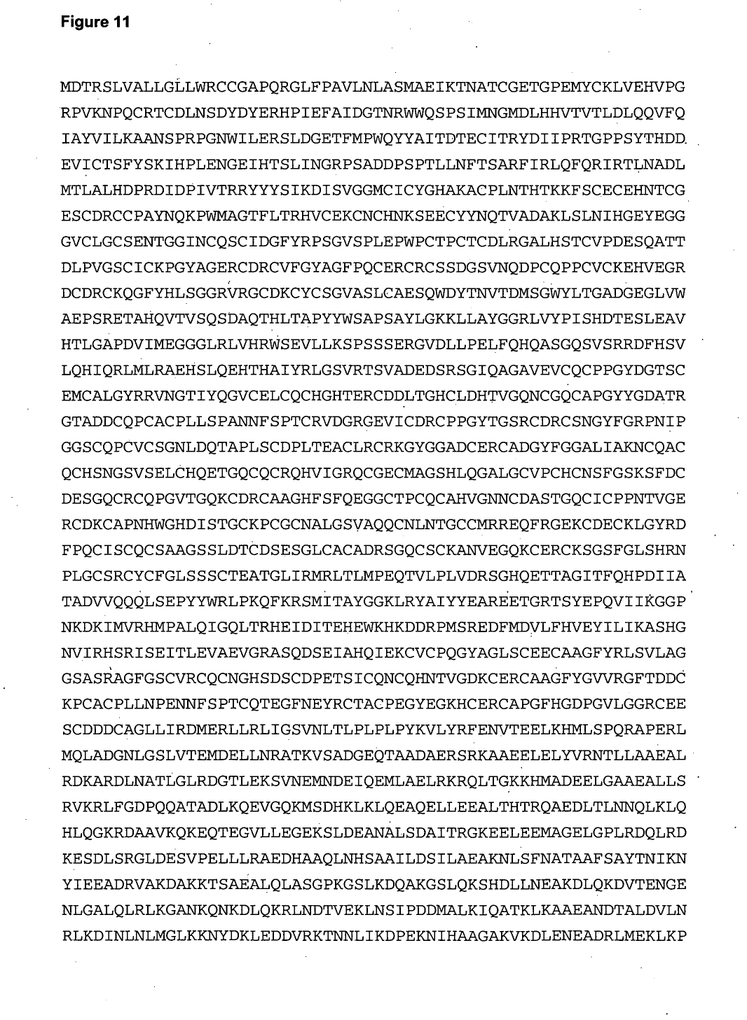

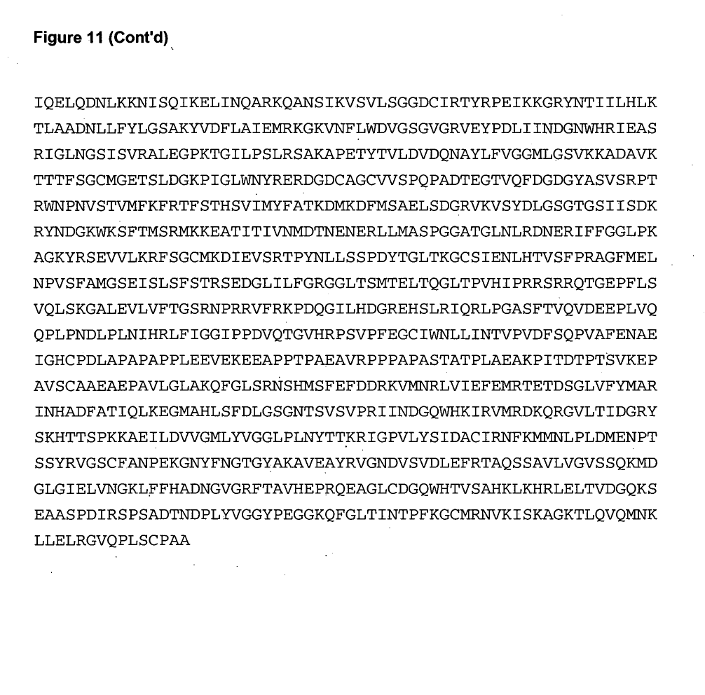

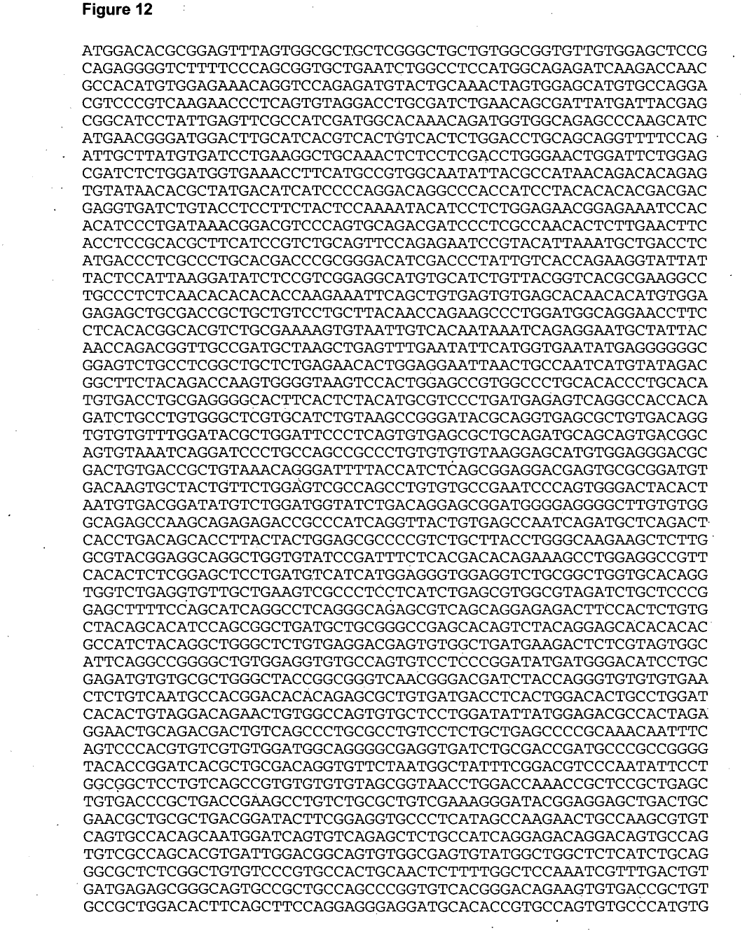

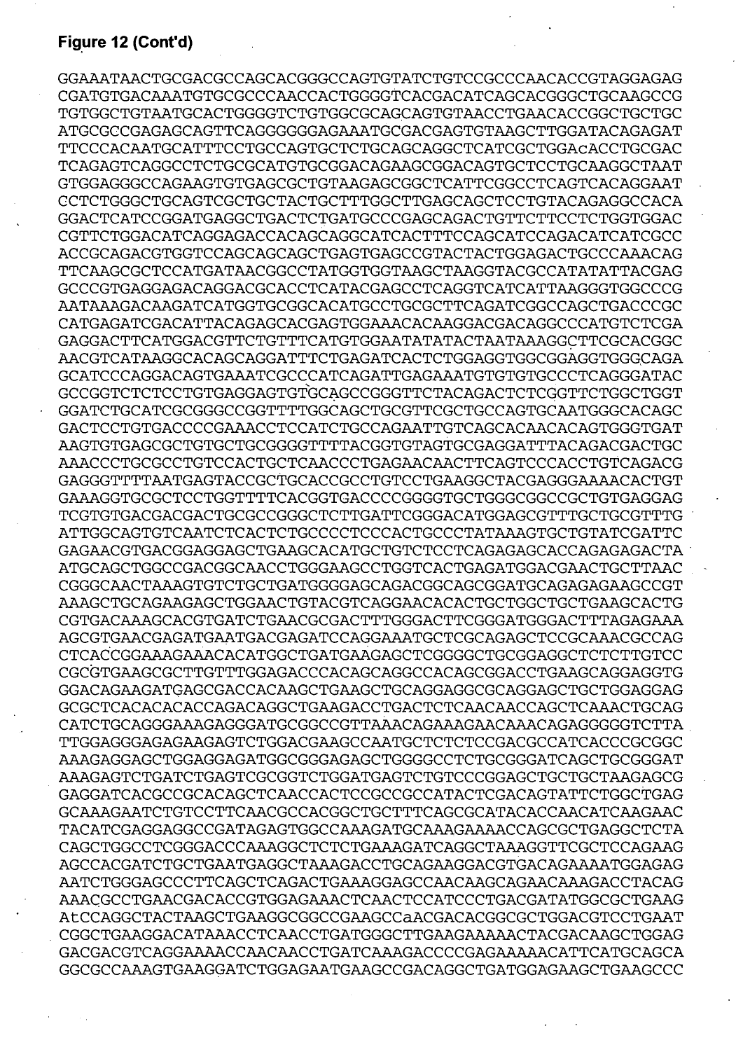

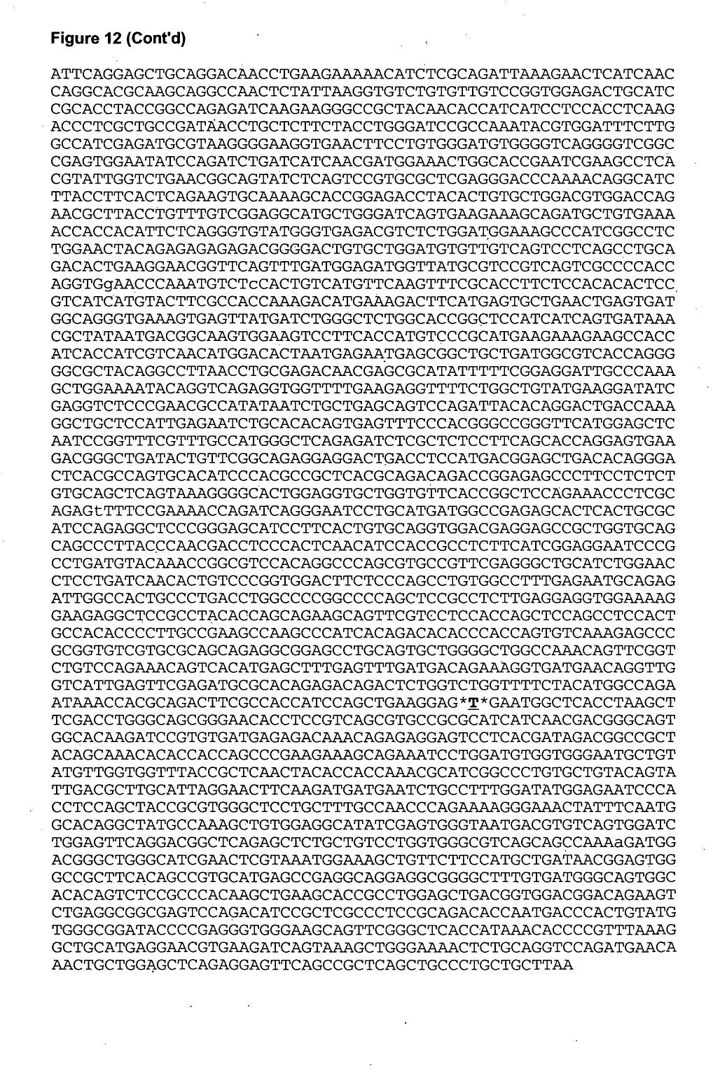

Under low powered EM (×2400) at 72 hpf, the thickness and architecture of the vertical myosepta were indistinguishable between mutant and sibs (FIG. 9). However, under higher magnification (×7100-x54000), tearing and detachment at the periphery were apparent in the mutants. By 120 hpf, the myoseptal architecture in the mutant embryos was grossly distorted and bubbled. Most significantly, portions of connective tissue were seen to infiltrate the myotome carried with the ends of retracting fibres. The myoseptum itself was greatly expanded in thickness, and contained an irregular array of collagen fibres.

### SUMMARY

A novel zebrafish model of laminin α2 deficient congenital muscular dystrophy has been developed. The present inventors have found that the cellular pathology in this model occurs by fibre detachment in the absence of catastrophic sarcolemmal failure. Also found is that innervation by the primary motor neurons is unaffected, and that early myoblast proliferation and fusion is normal.

It has been found that caf fish can be viable in the homozygous state, opening up the possibility of recessive screening for genetic modifiers of the lama2 locus. In addition, the capacity for regeneration suggests that screening against chemical libraries may provide insight into novel ameliorative pathways.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-17. (canceled)

18. A method for screening an agent having potential activity on muscular dystrophy comprising:
providing a fish model comprising an isolated zebrafish genetic strain having a laminin mutant phenotype resulting from a mutation within the zebrafish lama2 gene;
exposing the zebrafish to the agent; and
determining any effect of the agent on a genetic or physical characteristic of the zebrafish or its progeny.

19. The method according to claim 18 wherein the agent is a drug candidate, chemical, nucleic acid or compound.

20. The method according to claim 18 wherein the agent is administered by direct dilution in raising media, or direct administration to the fish.

21. The method according to claim 18 wherein muscular dystrophy is human congenital muscular dystrophy.

22. The method according to claim 18 wherein the effect is determined by visual or light microscopic technique selected from optical inspection of living muscle tissue, birefringency of muscle tissue using polarised light, use of fluorescent protein transgenic lines driven by muscle specific promoter(s), use of immunohistochemistry, use of antibodies directed against muscle specific epitopes, or in situ hybridisation for muscle specific gene expression.

23. The method according to claim 18 further comprising monitoring the effect of the agent on a genetic or physical characteristic of the zebrafish or its progeny.

24. The method according to claim 18 wherein the zebrafish has a candyfloss phenotype.

25. The method according to claim 24 wherein the candyfloss phenotype is caused by caf^{teg15a} or caf^{tk209}.

26. The method according claim 18 wherein the zebrafish includes progeny, fry, egg or gametes.

27. An isolated nucleic acid molecule encoding a mutation in the zebrafish lama2 gene forming a candyfloss phenotype.

28. The isolated nucleic acid molecule according to claim 27 wherein the candyfloss phenotype is caused by Caf^{teg15a} or caf^{tk209}.

29. The isolated nucleic acid molecule according to claim 28 encoding a lama2 mutation in zebrafish having a mutation as set out in SEQ ID NO: 3 or SEQ ID NO: 4.

30. An isolated nucleic acid molecule encoding lama2 gene of zebrafish or nucleic acid molecules complementary to the nucleic acid molecule encoding the lama2 gene, or nucleic acid molecules that hybridise under stringent conditions to the nucleic acid molecule encoding lama2 gene.

31. The isolated nucleic acid molecule according to claim 30 having the cDNA sequence substantially as set out in SEQ ID NO: 1.

32. An isolated zebrafish lama2 protein encoded by the nucleic acid molecule according to claim 30.

33. An isolated zebrafish lama2 protein encoded by the nucleic acid molecule according to claim 31.

34. The isolated protein according to claim 33 having the amino acid sequence substantially as set out in SEQ ID NO: 2.


SEQUENCE LISTING (4 sequences)

SEQ ID NO: 1 - DNA, 9228 bp, Zebrafish
atggacacgcggagtttagtggcgctgctcgggctgctgtggcggtgttgtggagctccgcagaggggtcttttcccagcggtgctgaatctggcctccatggcagagatcaagaccaacgccacatgtggagaaacaggtccagagatgtactgcaaactagtggagcatgtgccaggacgtcccgtcaagaaccctcagtgtaggacctgcgatctgaacagcgattatgattacgagcggcatcctattgagttcgccatcgatggcacaaacagatggtggcagagcccaagcatcatgaacgggatggacttgcatcacgtcactgtcactctggacctgcagcaggttttccagattgcttatgtgatcctgaaggctgcaaactctcctcgacctgggaactggattctggagcgatctctggatggtgaaaccttcatgccgtggcaatattacgccataacagacacagagtgtataacacgctatgacatcatccccaggacaggcccaccatcctacacacacgacgacgaggtgatctgtacctccttctactccaaaatacatcctctggagaacggagaaatccacacatccctgataaacggacgtcccagtgcagacgatccctcgccaacactcttgaacttcacctccgcacgcttcatccgtctgcagttccagagaatccgtacattaaatgctgacctcatgaccctcgccctgcacgacccgcgggacatcgaccctattgtcaccagaaggtattattactccattaaggatatctccgtcggaggcatgtgcatctgttacggtcacgcgaaggcctgccctctcaacacacacaccaagaaattcagctgtgagtgtgagcacaacacatgtggagagagctgcgaccgctgctgtcctgcttacaaccagaagccctggatggcaggaaccttcctcacacggcacgtctgcgaaaagtgtaattgtcacaataaatcagaggaatgctattacaaccagacggttgccgatgctaagctgagtttgaatattcatggtgaatatgaggggggcggagtctgcctcggctgctctgagaacactggaggaattaactgccaatcatgtatagacggcttctacagaccaagtggggtaagtccactggagccgtggccctgcacaccctgcacatgtgacctgcgaggggcacttcactctacatgcgtccctgatgagagtcaggccaccacagatctgcctgtgggctcgtgcatctgtaagccgggatacgcaggtgagcgctgtgacaggtgtgtgtttggatacgctggattccctcagtgtgagcgctgcagatgcagcagtgacggcagtgtaaatcaggatccctgccagccgccctgtgtgtgtaaggagcatgtggagggacgcgactgtgaccgctgtaaacagggattttaccatctcagcggaggacgagtgcgcggatgtgacaagtgctactgttctggagtcgccagcctgtgtgccgaatcccagtgggactacactaatgtgacggatatgtctggatggtatctgacaggagcggatggggaggggcttgtgtgggcagagccaagcagagagaccgcccatcaggttactgtgagccaatcagatgctcagactcacctgacagcaccttactactggagcgccccgtctgcttacctgggcaagaagctcttggcgtacggaggcaggctggtgtatccgatttctcacgacacagaaagcctggaggccgttcacactctcggagctcctgatgtcatcatggagggtggaggtctgcggctggtgcacaggtggtctgaggtgttgctgaagtcgccctcctcatctgagcgtggcgtagatctgctcccggagcttttccagcatcaggcctcagggcagagcgtcagcaggagagacttccactctgtgctacagcacatccagcggctgatgctgcgggccgagcacagtctacaggagcacacacacgccatctacaggctgggctctgtgaggacgagtgtggctgatgaagactctcgtagtggcattcaggccggggctgtggaggtgtgccagtgtcctcccggatatgatgggacatcctgcgagatgtgtgcgctgggctaccggcgggtcaacgggacgatctaccagggtgtgtgtgaactctgtcaatgccacggacacacagagcgctgtgatgacctcactggacactgcctggatcacactgtaggacagaactgtggccagtgtgctcctggatattatggagacgccactagaggaactgcagacgactgtcagccctgcgcctgtcctctgctgagccccgcaaacaatttcagtcccacgtgtcgtgtggatggcaggggcgaggtgatctgcgaccgatgcccgccggggtacaccggatcacgctgcgacaggtgttctaatggctatttcggacgtcccaatattcctggcggctcctgtcagccgtgtgtgtgtagcggtaacctggaccaaaccgctccgctgagctgtgacccgctgaccgaagcctgtctgcgctgtcgaaagggatacggaggagctgactgcgaacgctgcgctgacggatacttcggaggtgccctcatagccaagaactgccaagcgtgtcagtgccacagcaatggatcagtgtcagagctctgccatcaggagacaggacagtgccagtgtcgccagcacgtgattggacggcagtgtggcgagtgtatggctggctctcatctgcagggcgctctcggctgtgtcccgtgccactgcaactcttttggctccaaatcgtttgactgtgatgagagcgggcagtgccgctgccagcccggtgtcacgggacagaagtgtgaccgctgtgccgctggacacttcagcttccaggagggaggatgcacaccgtgccagtgtgcccatgtgggaaataactgcgacgccagcacgggccagtgtatctgtccgcccaacaccgtaggagagcgatgtgacaaatgtgcgcccaaccactggggtcacgacatcagcacgggctgcaagccgtgtggctgtaatgcactggggtctgtggcgcagcagtgtaacctgaacaccggctgctgcatgcgccgagagcagttcaggggggagaaatgcgacgagtgtaagcttggatacagagatttcccacaatgcatttcctgccagtgctctgcagcaggctcatcgctggacacctgcgactcagagtcaggcctctgcgcatgtgcggacagaagcggacagtgctcctgcaaggctaatgtggagggccagaagtgtgagcgctgtaagagcggctcattcggcctcagtcacaggaatcctctgggctgcagtcgctgctactgctttggcttgagcagctcctgtacagaggccacaggactcatccggatgaggctgactctgatgcccgagcagactgttcttcctctggtggaccgttctggacatcaggagaccacagcaggcatcactttccagcatccagacatcatcgccaccgcagacgtggtccagcagcagctgagtgagccgtactactggagactgcccaaacagttcaagcgctccatgataacggcctatggtggtaagctaaggtacgccatatattacgaggcccgtgaggagacaggacgcacctcatacgagcctcaggtcatcattaagggtggcccgaataaagacaagatcatggtgcggcacatgcctgcgcttcagatcggccagctgacccgccatgagatcgacattacagagcacgagtggaaacacaaggacgacaggcccatgtctcgagaggacttcatggacgttctgtttcatgtggaatatatactaataaaggcttcgcacggcaacgtcataaggcacagcaggatttctgagatcactctggaggtggcggaggtgggcagagcatcccaggacagtgaaatcgcccatcagattgagaaatgtgtgtgccctcagggatacgccggtctctcctgtgaggagtgtgcagccgggttctacagactctcggttctggctggtggatctgcatcgcgggccggttttggcagctgcgttcgctgccagtgcaatgggcacagcgactcctgtgaccccgaaacctccatctgccagaattgtcagcacaacacagtgggtgataagtgtgagcgctgtgctgcggggttttacggtgtagtgcgaggatttacagacgactgcaaaccctgcgcctgtccactgctcaaccctgagaacaacttcagtcccacctgtcagacggagggttttaatgagtaccgctgcaccgcctgtcctgaaggctacgagggaaaacactgtgaaaggtgcgctcctggttttcacggtgaccccggggtgctgggcggccgctgtgaggagtcgtgtgacgacgactgcgccgggctcttgattcgggacatggagcgtttgctgcgtttgattggcagtgtcaatctcactctgcccctcccactgccctataaagtgctgtatcgattcgagaacgtgacggaggagctgaagcacatgctgtctcctcagagagcaccagagagactaatgcagctggccgacggcaacctgggaagcctggtcactgagatggacgaactgcttaaccgggcaactaaagtgtctgctgatggggagcagacggcagcggatgcagagagaagccgtaaagctgcagaagagctggaactgtacgtcaggaacacactgctggctgctgaagcactgcgtgacaaagcacgtgatctgaacgcgactttgggacttcgggatgggactttagagaaaagcgtgaacgagatgaatgacgagatccaggaaatgctcgcagagctccgcaaacgccagctcaccggaaagaaacacatggctgatgaagagctcggggctgcggaggctctcttgtcccgcgtgaagcgcttgtttggagacccacagcaggccacagcggacctgaagcaggaggtgggacagaagatgagcgaccacaagctgaagctgcaggaggcgcaggagctgctggaggaggcgctcacacacaccagacaggctgaagacctgactctcaacaaccagctcaaactgcagcatctgcagggaaagagggatgcggccgttaaacagaaagaacaaacagagggggtcttattggagggagagaagagtctggacgaagccaatgctctctccgacgccatcacccgcggcaaagaggagctggaggagatggcgggagagctggggcctctgcgggatcagctgcgggataaagagtctgatctgagtcgcggtctggatgagtctgtcccggagctgctgctaagagcggaggatcacgccgcacagctcaaccactccgccgccatactcgacagtattctggctgaggcaaagaatctgtccttcaacgccacggctgctttcagcgcatacaccaacatcaagaactacatcgaggaggccgatagagtggccaaagatgcaaagaaaaccagcgctgaggctctacagctggcctcgggacccaaaggctctctgaaagatcaggctaaaggttcgctccagaagagccacgatctgctgaatgaggctaaagacctgcagaaggacgtgacagaaaatggagagaatctgggagcccttcagctcagactgaaaggagccaacaagcagaacaaagacctacagaaacgcctgaacgacaccgtggagaaactcaactccatccctgacgatatggcgctgaagatccaggctactaagctgaaggcggccgaagccaacgacacggcgctggacgtcctgaatcggctgaaggacataaacctcaacctgatgggcttgaagaaaaactacgacaagctggaggacgacgtcaggaaaaccaacaacctgatcaaagaccccgagaaaaacattcatgcagcaggcgccaaagtgaaggatctggagaatgaagccgacaggctgatggagaagctgaagcccattcaggagctgcaggacaacctgaagaaaaacatctcgcagattaaagaactcatcaaccaggcacgcaagcaggccaactctattaaggtgtctgtgttgtccggtggagactgcatccgcacctaccggccagagatcaagaagggccgctacaacaccatcatcctccacctcaagaccctcgctgccgataacctgctcttctacctgggatccgccaaatacgtggatttcttggccatcgagatgcgtaaggggaaggtgaacttcctgtgggatgtggggtcaggggtcggccgagtggaatatccagatctgatcatcaacgatggaaactggcaccgaatcgaagcctcacgtattggtctgaacggcagtatctcagtccgtgcgctcgagggacccaaaacaggcatcttaccttcactcagaagtgcaaaagcaccggagacctacactgtgctggacgtggaccagaacgcttacctgtttgtcggaggcatgctgggatcagtgaagaaagcagatgctgtgaaaaccaccacattctcagggtgtatgggtgagacgtctctggatggaaagcccatcggcctctggaactacagagagagagacggggactgtgctggatgtgttgtcagtcctcagcctgcagacactgaaggaacggttcagtttgatggagatggttatgcgtccgtcagtcgccccaccaggtggaacccaaatgtctccactgtcatgttcaagtttcgcaccttctccacacactccgtcatcatgtacttcgccaccaaagacatgaaagacttcatgagtgctgaactgagtgatggcagggtgaaagtgagttatgatctgggctctggcaccggctccatcatcagtgataaacgctataatgacggcaagtggaagtccttcaccatgtcccgcatgaagaaagaagccaccatcaccatcgtcaacatggacactaatgagaatgagcggctgctgatggcgtcaccagggggcgctacaggccttaacctgcgagacaacgagcgcatatttttcggaggattgcccaaagctggaaaatacaggtcagaggtggttttgaagaggttttctggctgtatgaaggatatcgaggtctcccgaacgccatataatctgctgagcagtccagattacacaggactgaccaaaggctgctccattgagaatctgcacacagtgagtttcccacgggccgggttcatggagctcaatccggtttcgtttgccatgggctcagagatctcgctctccttcagcaccaggagtgaagacgggctgatactgttcggcagaggaggactgacctccatgacggagctgacacagggactcacgccagtgcacatcccacgccgctcacgcagacagaccggagagcccttcctctctgtgcagctcagtaaaggggcactggaggtgctggtgttcaccggctccagaaaccctcgcagagttttccgaaaaccagatcagggaatcctgcatgatggccgagagcactcactgcgcatccagaggctcccgggagcatccttcactgtgcaggtggacgaggagccgctggtgcagcagcccttacccaacgacctcccactcaacatccaccgcctcttcatcggaggaatcccgcctgatgtacaaaccggcgtccacaggcccagcgtgccgttcgagggctgcatctggaacctcctgatcaacactgtcccggtggacttctcccagcctgtggcctttgagaatgcagagattggccactgccctgacctggccccggccccagctccgcctcttgaggaggtggaaaaggaagaggctccgcctacaccagcagaagcagttcgtcctccaccagctccagcctccactgccacaccccttgccgaagccaagcccatcacagacacacccaccagtgtcaaagagcccgcggtgtcgtgcgcagcagaggcggagcctgcagtgctggggctggccaaacagttcggtctgtccagaaacagtcacatgagctttgagtttgatgacagaaaggtgatgaacaggttggtcattgagttcgagatgcgcacagagacagactctggtctggttttctacatggccagaataaaccacgcagacttcgccaccatccagctgaaggagggaatggctcacctaagcttcgacctgggcagcgggaacacctccgtcagcgtgccgcgcatcatcaacgacgggcagtggcacaagatccgtgtgatgagagacaaacagagaggagtcctcacgatagacggccgctacagcaaacacaccaccagcccgaagaaagcagaaatcctggatgtggtgggaatgctgtatgttggtggtttaccgctcaactacaccaccaaacgcatcggccctgtgctgtacagtattgacgcttgcattaggaacttcaagatgatgaatctgcctttggatatggagaatcccacctccagctaccgcgtgggctcctgctttgccaacccagaaaagggaaactatttcaatggcacaggctatgccaaagctgtggaggcatatcgagtgggtaatgacgtgtcagtggatctggagttcaggacggctcagagctctgctgtcctggtgggcgtcagcagccaaaagatggacgggctgggcatcgaactcgtaaatggaaagctgttcttccatgctgataacggagtgggccgcttcacagccgtgcatgagccgaggcaggaggcggggctttgtgatgggcagtggcacacagtctccgcccacaagctgaagcaccgcctggagctgacggtggacggacagaagtctgaggcggcgagtccagacatccgctcgccctccgcagacaccaatgacccactgtatgtgggcggataccccgagggtgggaagcagttcgggctcaccataaacaccccgtttaaaggctgcatgaggaacgtgaagatcagtaaagctgggaaaactctgcaggtccagatgaacaaactgctggagctcagaggagttcagccgctcagctgccctgctgcttaa

SEQ ID NO: 2 - protein, 3075 aa, Zebrafish
MDTRSLVALLGLLWRCCGAPQRGLFPAVLNLASMAEIKTNATCGETGPEMYCKLVEHVPGRPVKNPQCRTCDLNSDYDYERHPIEFAIDGTNRWWQSPSIMNGMDLHHVTVTLDLQQVFQIAYVILKAANSPRPGNWILERSLDGETFMPWQYYAITDTECITRYDIIPRTGPPSYTHDDEVICTSFYSKIHPLENGEIHTSLINGRPSADDPSPTLLNFTSARFIRLQFQRIRTLNADLMTLALHDPRDIDPIVTRRYYYSIKDISVGGMCICYGHAKACPLNTHTKKFSCECEHNTCGESCDRCCPAYNQKPWMAGTFLTRHVCEKCNCHNKSEECYYNQTVADAKLSLNIHGEYEGGGVCLGCSENTGGINCQSCIDGFYRPSGVSPLEPWPCTPCTCDLRGALHSTCVPDESQATTDLPVGSCICKPGYAGERCDRCVFGYAGFPQCERCRCSSDGSVNQDPCQPPCVCKEHVEGRDCDRCKQGFYHLSGGRVRGCDKCYCSGVASLCAESQWDYTNVTDMSGWYLTGADGEGLVWAEPSRETAHQVTVSQSDAQTHLTAPYYWSAPSAYLGKKLLAYGGRLVYPISHDTESLEAVHTLGAPDVIMEGGGLRLVHRWSEVLLKSPSSSERGVDLLPELFQHQASGQSVSRRDFHSVLQHIQRLMLRAEHSLQEHTHAIYRLGSVRTSVADEDSRSGIQAGAVEVCQCPPGYDGTSCEMCALGYRRVNGTIYQGVCELCQCHGHTERCDDLTGHCLDHTVGQNCGQCAPGYYGDATRGTADDCQPCACPLLSPANNFSPTCRVDGRGEVICDRCPPGYTGSRCDRCSNGYFGRPNIPGGSCQPCVCSGNLDQTAPLSCDPLTEACLRCRKGYGGADCERCADGYFGGALIAKNCQACQCHSNGSVSELCHQETGQCQCRQHVIGRQCGECMAGSHLQGALGCVPCHCNSFGSKSFDCDESGQCRCQPGVTGQKCDRCAAGHFSFQEGGCTPCQCAHVGNNCDASTGQCICPPNTVGERCDKCAPNHWGHDISTGCKPCGCNALGSVAQQCNLNTGCCMRREQFRGEKCDECKLGYRDFPQCISCQCSAAGSSLDTCDSESGLCACADRSGQCSCKANVEGQKCERCKSGSFGLSHRNPLGCSRCYCFGLSSSCTEATGLIRMRLTLMPEQTVLPLVDRSGHQETTAGITFQHPDIIATADVVQQQLSEPYYWRLPKQFKRSMITAYGGKLRYAIYYEAREETGRTSYEPQVIIKGGPNKDKIMVRHMPALQIGQLTRHEIDITEHEWKHKDDRPMSREDFMDVLFHVEYILIKASHGNVIRHSRISEITLEVAEVGRASQDSEIAHQIEKCVCPQGYAGLSCEECAAGFYRLSVLAGGSASRAGFGSCVRCQCNGHSDSCDPETSICQNCQHNTVGDKCERCAAGFYGVVRGFTDDCKPCACPLLNPENNFSPTCQTEGFNEYRCTACPEGYEGKHCERCAPGFHGDPGVLGGRCEESCDDDCAGLLIRDMERLLRLIGSVNLTLPLPLPYKVLYRFENVTEELKHMLSPQRAPERLMQLADGNLGSLVTEMDELLNRATKVSADGEQTAADAERSRKAAEELELYVRNTLLAAEALRDKARDLNATLGLRDGTLEKSVNEMNDEIQEMLAELRKRQLTGKKHMADEELGAAEALLSRVKRLFGDPQQATADLKQEVGQKMSDHKLKLQEAQELLEEALTHTRQAEDLTLNNQLKLQHLQGKRDAAVKQKEQTEGVLLEGEKSLDEANALSDAITRGKEELEEMAGELGPLRDQLRDKESDLSRGLDESVPELLLRAEDHAAQLNHSAAILDSILAEAKNLSFNATAAFSAYTNIKNYIEEADRVAKDAKKTSAEALQLASGPKGSLKDQAKGSLQKSHDLLNEAKDLQKDVTENGENLGALQLRLKGANKQNKDLQKRLNDTVEKLNSIPDDMALKIQATKLKAAEANDTALDVLNRLKDINLNLMGLKKNYDKLEDDVRKTNNLIKDPEKNIHAAGAKVKDLENEADRLMEKLKPIQELQDNLKKNISQIKELINQARKQANSIKVSVLSGGDCIRTYRPEIKKGRYNTIILHLKTLAADNLLFYLGSAKYVDFLAIEMRKGKVNFLWDVGSGVGRVEYPDLIINDGNWHRIEASRIGLNGSISVRALEGPKTGILPSLRSAKAPETYTVLDVDQNAYLFVGGMLGSVKKADAVKTTTFSGCMGETSLDGKPIGLWNYRERDGDCAGCVVSPQPADTEGTVQFDGDGYASVSRPTRWNPNVSTVMFKFRTFSTHSVIMYFATKDMKDFMSAELSDGRVKVSYDLGSGTGSIISDKRYNDGKWKSFTMSRMKKEATITIVNMDTNENERLLMASPGGATGLNLRDNERIFFGGLPKAGKYRSEVVLKRFSGCMKDIEVSRTPYNLLSSPDYTGLTKGCSIENLHTVSFPRAGFMELNPVSFAMGSEISLSFSTRSEDGLILFGRGGLTSMTELTQGLTPVHIPRRSRRQTGEPFLSVQLSKGALEVLVFTGSRNPRRVFRKPDQGILHDGREHSLRIQRLPGASFTVQVDEEPLVQQPLPNDLPLNIHRLFIGGIPPDVQTGVHRPSVPFEGCIWNLLINTVPVDFSQPVAFENAEIGHCPDLAPAPAPPLEEVEKEEAPPTPAEAVRPPPAPASTATPLAEAKPITDTPTSVKEPAVSCAAEAEPAVLGLAKQFGLSRNSHMSFEFDDRKVMNRLVIEFEMRTETDSGLVFYMARINHADFATIQLKEGMAHLSFDLGSGNTSVSVPRIINDGQWHKIRVMRDKQRGVLTIDGRYSKHTTSPKKAEILDVVGMLYVGGLPLNYTTKRIGPVLYSIDACIRNFKMMNLPLDMENPTSSYRVGSCFANPEKGNYFNGTGYAKAVEAYRVGNDVSVDLEFRTAQSSAVLVGVSSQKMDGLGIELVNGKLFFHADNGVGRFTAVHEPRQEAGLCDGQWHTVSAHKLKHRLELTVDGQKSEAASPDIRSPSADTNDPLYVGGYPEGGKQFGLTINTPFKGCMRNVKISKAGKTLQVQMNKLLELRGVQPLSCPAA

SEQ ID NO: 3 - DNA, 9228 bp, Zebrafish
atggacacgcggagtttagtggcgctgctcgggctgctgtggcggtgttgtggagctccgcagaggggtcttttcccagcggtgctgaatctggcctccatggcagagatcaagaccaacgccacatgtggagaaacaggtccagagatgtactgcaaactagtggagcatgtgccaggacgtcccgtcaagaaccctcagtgtaggacctgcgatctgaacagcgattatgattacgagcggcatcctattgagttcgccatcgatggcacaaacagatggtggcagagcccaagcatcatgaacgggatggacttgcatcacgtcactgtcactctggacctgcagcaggttttccagattgcttatgtgatcctgaaggctgcaaactctcctcgacctgggaactggattctggagcgatctctggatggtgaaaccttcatgccgtggcaatattacgccataacagacacagagtgtataacacgctatgacatcatccccaggacaggcccaccatcctacacacacgacgacgaggtgatctgtacctccttctactccaaaatacatcctctggagaacggagaaatccacacatccctgataaacggacgtcccagtgcagacgatccctcgccaacactcttgaacttcacctccgcacgcttcatccgtctgcagttccagagaatccgtacattaaatgctgacctcatgaccctcgccctgcacgacccgcgggacatcgaccctattgtcaccagaaggtattattactccattaaggatatctccgtcggaggcatgtgcatctgttacggtcacgcgaaggcctgccctctcaacacacacaccaagaaattcagctgtgagtgtgagcacaacacatgtggagagagctgcgaccgctgctgtcctgcttacaaccagaagccctggatggcaggaaccttcctcacacggcacgtctgcgaaaagtgtaattgtcacaataaatcagaggaatgctattacaaccagacggttgccgatgctaagctgagtttgaatattcatggtgaatatgaggggggcggagtctgcctcggctgctctgagaacactggaggaattaactgccaatcatgtatagacggcttctacagaccaagtggggtaagtccactggagccgtggccctgcacaccctgcacatgtgacctgcgaggggcacttcactctacatgcgtccctgatgagagtcaggccaccacagatctgcctgtgggctcgtgcatctgtaagccgggatacgcaggtgagcgctgtgacaggtgtgtgtttggatacgctggattccctcagtgtgagcgctgcagatgcagcagtgacggcagtgtaaatcaggatccctgccagccgccctgtgtgtgtaaggagcatgtggagggacgcgactgtgaccgctgtaaacagggattttaccatctcagcggaggacgagtgcgcggatgtgacaagtgctactgttctggagtcgccagcctgtgtgccgaatcccagtgggactacactaatgtgacggatatgtctggatggtatctgacaggagcggatggggaggggcttgtgtgggcagagccaagcagagagaccgcccatcaggttactgtgagccaatcagatgctcagactcacctgacagcaccttactactggagcgccccgtctgcttacctgggcaagaagctcttggcgtacggaggcaggctggtgtatccgatttctcacgacacagaaagcctggaggccgttcacactctcggagctcctgatgtcatcatggagggtggaggtctgcggctggtgcacaggtggtctgaggtgttgctgaagtcgccctcctcatctgagcgtggcgtagatctgctcccggagcttttccagcatcaggcctcagggcagagcgtcagcaggagagacttccactctgtgctacagcacatccagcggctgatgctgcgggccgagcacagtctacaggagcacacacacgccatctacaggctgggctctgtgaggacgagtgtggctgatgaagactctcgtagtggcattcaggccggggctgtggaggtgtgccagtgtcctcccggatatgatgggacatcctgcgagatgtgtgcgctgggctaccggcgggtcaacgggacgatctaccagggtgtgtgtgaactctgtcaatgccacggacacacagagcgctgtgatgacctcactggacactgcctggatcacactgtaggacagaactgtggccagtgtgctcctggatattatggagacgccactagaggaactgcagacgactgtcagccctgcgcctgtcctctgctgagccccgcaaacaatttcagtcccacgtgtcgtgtggatggcaggggcgaggtgatctgcgaccgatgcccgccggggtacaccggatcacgctgcgacaggtgttctaatggctatttcggacgtcccaatattcctggcggctcctgtcagccgtgtgtgtgtagcggtaacctggaccaaaccgctccgctgagctgtgacccgctgaccgaagcctgtctgcgctgtcgaaagggatacggaggagctgactgcgaacgctgcgctgacggatacttcggaggtgccctcatagccaagaactgccaagcgtgtcagtgccacagcaatggatcagtgtcagagctctgccatcaggagacaggacagtgccagtgtcgccagcacgtgattggacggcagtgtggcgagtgtatggctggctctcatctgcagggcgctctcggctgtgtcccgtgccactgcaactcttttggctccaaatcgtttgactgtgatgagagcgggcagtgccgctgccagcccggtgtcacgggacagaagtgtgaccgctgtgccgctggacacttcagcttccaggagggaggatgcacaccgtgccagtgtgcccatgtgggaaataactgcgacgccagcacgggccagtgtatctgtccgcccaacaccgtaggagagcgatgtgacaaatgtgcgcccaaccactggggtcacgacatcagcacgggctgcaagccgtgtggctgtaatgcactggggtctgtggcgcagcagtgtaacctgaacaccggctgctgcatgcgccgagagcagttcaggggggagaaatgcgacgagtgtaagcttggatacagagatttcccacaatgcatttcctgccagtgctctgcagcaggctcatcgctggacacctgcgactcagagtcaggcctctgcgcatgtgcggacagaagcggacagtgctcctgcaaggctaatgtggagggccagaagtgtgagcgctgtaagagcggctcattcggcctcagtcacaggaatcctctgggctgcagtcgctgctactgctttggcttgagcagctcctgtacagaggccacaggactcatccggatgaggctgactctgatgcccgagcagactgttcttcctctggtggaccgttctggacatcaggagaccacagcaggcatcactttccagcatccagacatcatcgccaccgcagacgtggtccagcagcagctgagtgagccgtactactggagactgcccaaacagttcaagcgctccatgataacggcctatggtggtaagctaaggtacgccatatattacgaggcccgtgaggagacaggacgcacctcatacgagcctcaggtcatcattaagggtggcccgaataaagacaagatcatggtgcggcacatgcctgcgcttcagatcggccagctgacccgccatgagatcgacattacagagcacgagtggaaacacaaggacgacaggcccatgtctcgagaggacttcatggacgttctgtttcatgtggaatatatactaataaaggcttcgcacggcaacgtcataaggcacagcaggatttctgagatcactctggaggtggcggaggtgggcagagcatcccaggacagtgaaatcgcccatcagattgagaaatgtgtgtgccctcagggatacgccggtctctcctgtgaggagtgtgcagccgggttctacagactctcggttctggctggtggatctgcatcgcgggccggttttggcagctgcgttcgctgccagtgcaatgggcacagcgactcctgtgaccccgaaacctccatctgccagaattgtcagcacaacacagtgggtgataagtgtgagcgctgtgctgcggggttttacggtgtagtgcgaggatttacagacgactgcaaaccctgcgcctgtccactgctcaaccctgagaacaacttcagtcccacctgtcagacggagggttttaatgagtaccgctgcaccgcctgtcctgaaggctacgagggaaaacactgtgaaaggtgcgctcctggttttcacggtgaccccggggtgctgggcggccgctgtgaggagtcgtgtgacgacgactgcgccgggctcttgattcgggacatggagcgtttgctgcgtttgattggcagtgtcaatctcactctgcccctcccactgccctataaagtgctgtatcgattcgagaacgtgacggaggagctgaagcacatgctgtctcctcagagagcaccagagagactaatgcagctggccgacggcaacctgggaagcctggtcactgagatggacgaactgcttaaccgggcaactaaagtgtctgctgatggggagcagacggcagcggatgcagagagaagccgtaaagctgcagaagagctggaactgtacgtcaggaacacactgctggctgctgaagcactgcgtgacaaagcacgtgatctgaacgcgactttgggacttcgggatgggactttagagaaaagcgtgaacgagatgaatgacgagatccaggaaatgctcgcagagctccgcaaacgccagctcaccggaaagaaacacatggctgatgaagagctcggggctgcggaggctctcttgtcccgcgtgaagcgcttgtttggagacccacagcaggccacagcggacctgaagcaggaggtgggacagaagatgagcgaccacaagctgaagctgcaggaggcgcaggagctgctggaggaggcgctcacacacaccagacaggctgaagacctgactctcaacaaccagctcaaactgcagcatctgcagggaaagagggatgcggccgttaaacagaaagaacaaacagagggggtcttattggagggagagaagagtctggacgaagccaatgctctctccgacgccatcacccgcggcaaagaggagctggaggagatggcgggagagctggggcctctgcgggatcagctgcgggataaagagtctgatctgagtcgcggtctggatgagtctgtcccggagctgctgctaagagcggaggatcacgccgcacagctcaaccactccgccgccatactcgacagtattctggctgaggcaaagaatctgtccttcaacgccacggctgctttcagcgcatacaccaacatcaagaactacatcgaggaggccgatagagtggccaaagatgcaaagaaaaccagcgctgaggctctacagctggcctcgggacccaaaggctctctgaaagatcaggctaaaggttcgctccagaagagccacgatctgctgaatgaggctaaagacctgcagaaggacgtgacagaaaatggagagaatctgggagcccttcagctcagactgaaaggagccaacaagcagaacaaagacctacagaaacgcctgaacgacaccgtggagaaactcaactccatccctgacgatatggcgctgaagatccaggctactaagctgaaggcggccgaagccaacgacacggcgctggacgtcctgaatcggctgaaggacataaacctcaacctgatgggcttgaagaaaaactacgacaagctggaggacgacgtcaggaaaaccaacaacctgatcaaagaccccgagaaaaacattcatgcagcaggcgccaaagtgaaggatctggagaatgaagccgacaggctgatggagaagctgaagcccattcaggagctgcaggacaacctgaagaaaaacatctcgcagattaaagaactcatcaaccaggcacgcaagcaggccaactctattaaggtgtctgtgttgtccggtggagactgcatccgcacctaccggccagagatcaagaagggccgctacaacaccatcatcctccacctcaagaccctcgctgccgataacctgctcttctacctgggatccgccaaatacgtggatttcttggccatcgagatgcgtaaggggaaggtgaacttcctgtgggatgtggggtcaggggtcggccgagtggaatatccagatctgatcatcaacgatggaaactggcaccgaatcgaagcctcacgtattggtctgaacggcagtatctcagtccgtgcgctcgagggacccaaaacaggcatcttaccttcactcagaagtgcaaaagcaccggagacctacactgtgctggacgtggaccagaacgcttacctgtttgtcggaggcatgctgggatcagtgaagaaagcagatgctgtgaaaaccaccacattctcagggtgtatgggtgagacgtctctggatggaaagcccatcggcctctggaactacagagagagagacggggactgtgctggatgtgttgtcagtcctcagcctgcagacactgaaggaacggttcagtttgatggagatggttatgcgtccgtcagtcgccccaccaggtggaacccaaatgtctccactgtcatgttcaagtttcgcaccttctccacacactccgtcatcatgtacttcgccaccaaagacatgaaagacttcatgagtgctgaactgagtgatggcagggtgaaagtgagttatgatctgggctctggcaccggctccatcatcagtgataaacgctataatgacggcaagtggaagtccttcaccatgtcccgcatgaagaaagaagccaccatcaccatcgtcaacatggacactaatgagaatgagcggctgctgatggcgtcaccagggggcgctacaggccttaacctgcgagacaacgagcgcatatttttcggaggattgcccaaagctggaaaatacaggtcagaggtggttttgaagaggttttctggctgtatgaaggatatcgaggtctcccgaacgccatataatctgctgagcagtccagattacacaggactgaccaaaggctgctccattgagaatctgcacacagtgagtttcccacgggccgggttcatggagctcaatccggtttcgtttgccatgggctcagagatctcgctctccttcagcaccaggagtgaagacgggctgatactgttcggcagaggaggactgacctccatgacggagctgacacagggactcacgccagtgcacatcccacgccgctcacgcagacagaccggagagcccttcctctctgtgcagctcagtaaaggggcactggaggtgctggtgttcaccggctccagaaaccctcgcagagttttccgaaaaccagatcagggaatcctgcatgatggccgagagcactcactgcgcatccagaggctcccgggagcatccttcactgtgcaggtggacgaggagccgctggtgcagcagcccttacccaacgacctcccactcaacatccaccgcctcttcatcggaggaatcccgcctgatgtacaaaccggcgtccacaggcccagcgtgccgttcgagggctgcatctggaacctcctgatcaacactgtcccggtggacttctcccagcctgtggcctttgagaatgcagagattggccactgccctgacctggccccggccccagctccgcctcttgaggaggtggaaaaggaagaggctccgcctacaccagcagaagcagttcgtcctccaccagctccagcctccactgccacaccccttgccgaagccaagcccatcacagacacacccaccagtgtcaaagagcccgcggtgtcgtgcgcagcagaggcggagcctgcagtgctggggctggccaaacagttcggtctgtccagaaacagtcacatgagctttgagtttgatgacagaaaggtgatgaacaggttggtcattgagttcgagatgcgcacagagacagactctggtctggttttctacatggccagaataaaccacgcagacttcgccaccatccagctgaaggagtgaatggctcacctaagcttcgacctgggcagcgggaacacctccgtcagcgtgccgcgcatcatcaacgacgggcagtggcacaagatccgtgtgatgagagacaaacagagaggagtcctcacgatagacggccgctacagcaaacacaccaccagcccgaagaaagcagaaatcctggatgtggtgggaatgctgtatgttggtggtttaccgctcaactacaccaccaaacgcatcggccctgtgctgtacagtattgacgcttgcattaggaacttcaagatgatgaatctgcctttggatatggagaatcccacctccagctaccgcgtgggctcctgctttgccaacccagaaaagggaaactatttcaatggcacaggctatgccaaagctgtggaggcatatcgagtgggtaatgacgtgtcagtggatctggagttcaggacggctcagagctctgctgtcctggtgggcgtcagcagccaaaagatggacgggctgggcatcgaactcgtaaatggaaagctgttcttccatgctgataacggagtgggccgcttcacagccgtgcatgagccgaggcaggaggcggggctttgtgatgggcagtggcacacagtctccgcccacaagctgaagcaccgcctggagctgacggtggacggacagaagtctgaggcggcgagtccagacatccgctcgccctccgcagacaccaatgacccactgtatgtgggcggataccccgagggtgggaagcagttcgggctcaccataaacaccccgtttaaaggctgcatgaggaacgtgaagatcagtaaagctgggaaaactctgcaggtccagatgaacaaactgctggagctcagaggagttcagccgctcagctgccctgctgcttaa

SEQ ID NO: 4 - DNA, 9228 bp, Zebrafish
atggacacgcggagtttagtggcgctgctcgggctgctgtggcggtgttgtggagctccgcagaggggtcttttcccagcggtgctgaatctggcctccatggcagagatcaagaccaacgccacatgtggagaaacaggtccagagatgtactgcaaactagtggagcatgtgccaggacgtcccgtcaagaaccctcagtgtaggacctgcgatctgaacagcgattatgattacgagcggcatcctattgagttcgccatcgatggcacaaacagatggtggcagagcccaagcatcatgaacgggatggacttgcatcacgtcactgtcactctggacctgcagcaggttttccagattgcttatgtgatcctgaaggctgcaaactctcctcgacctgggaactggattctggagcgatctctggatggtgaaaccttcatgccgtggcaatattacgccataacagacacagagtgtataacacgctatgacatcatccccaggacaggcccaccatcctacacacacgacgacgaggtgatctgtacctccttctactccaaaatacatcctctggagaacggagaaatccacacatccctgataaacggacgtcccagtgcagacgatccctcgccaacactcttgaacttcacctccgcacgcttcatccgtctgcagttccagagaatccgtacattaaatgctgacctcatgaccctcgccctgcacgacccgcgggacatcgaccctattgtcaccagaaggtattattactccattaaggatatctccgtcggaggcatgtgcatctgttacggtcacgcgaaggcctgccctctcaacacacacaccaagaaattcagctgtgagtgtgagcacaacacatgtggagagagctgcgaccgctgctgtcctgcttacaaccagaagccctggatggcaggaaccttcctcacacggcacgtctgcgaaaagtgtaattgtcacaataaatcagaggaatgctattacaaccagacggttgccgatgctaagctgagtttgaatattcatggtgaatatgaggggggcggagtctgcctcggctgctctgagaacactggaggaattaactgccaatcatgtatagacggcttctacagaccaagtggggtaagtccactggagccgtggccctgcacaccctgcacatgtgacctgcgaggggcacttcactctacatgcgtccctgatgagagtcaggccaccacagatctgcctgtgggctcgtgcatctgtaagccgggatacgcaggtgagcgctgtgacaggtgtgtgtttggatacgctggattccctcagtgtgagcgctgcagatgcagcagtgacggcagtgtaaatcaggatccctgccagccgccctgtgtgtgtaaggagcatgtggagggacgcgactgtgaccgctgtaaacagggattttaccatctcagcggaggacgagtgcgcggatgtgacaagtgctactgttctggagtcgccagcctgtgtgccgaatcccagtgggactacactaatgtgacggatatgtctggatggtatctgacaggagcggatggggaggggcttgtgtgggcagagccaagcagagagaccgcccatcaggttactgtgagccaatcagatgctcagactcacctgacagcaccttactactggagcgccccgtctgcttacctgggcaagaagctcttggcgtacggaggcaggctggtgtatccgatttctcacgacacagaaagcctggaggccgttcacactctcggagctcctgatgtcatcatggagggtggaggtctgcggctggtgcacaggtggtctgaggtgttgctgaagtcgccctcctcatctgagcgtggcgtagatctgctcccggagcttttccagcatcaggcctcagggcagagcgtcagcaggagagacttccactctgtgctacagcacatccagcggctgatgctgcgggccgagcacagtctacaggagcacacacacgccatctacaggctgggctctgtgaggacgagtgtggctgatgaagactctcgtagtggcattcaggccggggctgtggaggtgtgccagtgtcctcccggatatgatgggacatcctgcgagatgtgtgcgctgggctaccggcgggtcaacgggacgatctaccagggtgtgtgtgaactctgtcaatgccacggacacacagagcgctgtgatgacctcactggacactgcctggatcacactgtaggacagaactgtggccagtgtgctcctggatattatggagacgccactagaggaactgcagacgactgtcagccctgcgcctgtcctctgctgagccccgcaaacaatttcagtcccacgtgtcgtgtggatggcaggggcgaggtgatctgcgaccgatgcccgccggggtacaccggatcacgctgcgacaggtgttctaatggctatttcggacgtcccaatattcctggcggctcctgtcagccgtgtgtgtgtagcggtaacctggaccaaaccgctccgctgagctgtgacccgctgaccgaagcctgtctgcgctgtcgaaagggatacggaggagctgactgcgaacgctgcgctgacggatacttcggaggtgccctcatagccaagaactgccaagcgtgtcagtgccacagcaatggatcagtgtcagagctctgccatcaggagacaggacagtgccagtgtcgccagcacgtgattggacggcagtgtggcgagtgtatggctggctctcatctgcagggcgctctcggctgtgtcccgtgccactgcaactcttttggctccaaatcgtttgactgtgatgagagcgggcagtgccgctgccagcccggtgtcacgggacagaagtgtgaccgctgtgccgctggacacttcagcttccaggagggaggatgcacaccgtgccagtgtgcccatgtgggaaataactgcgacgccagcacgggccagtgtatctgtccgcccaacaccgtaggagagcgatgtgacaaatgtgcgcccaaccactggggtcacgacatcagcacgggctgcaagccgtgtggctgtaatgcactggggtctgtggcgcagcagtgtaacctgaacaccggctgctgcatgcgccgagagcagttcaggggggagaaatgcgacgagtgtaagcttggatacagagatttcccacaatgcatttcctgccagtgctctgcagcaggctcatcgctggacacctgcgactcagagtcaggcctctgcgcatgtgcggacagaagcggacagtgctcctgcaaggctaatgtggagggccagaagtgtgagcgctgtaagagcggctcattcggcctcagtcacaggaatcctctgggctgcagtcgctgctactgctttggcttgagcagctcctgtacagaggccacaggactcatccggatgaggctgactctgatgcccgagcagactgttcttcctctggtggaccgttctggacatcaggagaccacagcaggcatcactttccagcatccagacatcatcgccaccgcagacgtggtccagcagcagctgagtgagccgtactactggagactgcccaaacagttcaagcgctccatgataacggcctatggtggtaagctaaggtacgccatatattacgaggcccgtgaggagacaggacgcacctcatacgagcctcaggtcatcattaagggtggcccgaataaagacaagatcatggtgcggcacatgcctgcgcttcagatcggccagctgacccgccatgagatcgacattacagagcacgagtggaaacacaaggacgacaggcccatgtctcgagaggacttcatggacgttctgtttcatgtggaatatatactaataaaggcttcgcacggcaacgtcataaggcacagcaggatttctgagatcactctggaggtggcggaggtgggcagagcatcccaggacagtgaaatcgcccatcagattgagaaatgtgtgtgccctcagggatacgccggtctctcctgtgaggagtgtgcagccgggttctacagactctcggttctggctggtggatctgcatcgcgggccggttttggcagctgcgttcgctgccagtgcaatgggcacagcgactcctgtgaccccgaaacctccatctgccagaattgtcagcacaacacagtgggtgataagtgtgagcgctgtgctgcggggttttacggtgtagtgcgaggatttacagacgactgcaaaccctgcgcctgtccactgctcaaccctgagaacaacttcagtcccacctgtcagacggagggttttaatgagtaccgctgcaccgcctgtcctgaaggctacgagggaaaacactgtgaaaggtgcgctcctggttttcacggtgaccccggggtgctgggcggccgctgtgaggagtcgtgtgacgacgactgcgccgggctcttgattcgggacatggagcgtttgctgcgtttgattggcagtgtcaatctcactctgcccctcccactgccctataaagtgctgtatcgattcgagaacgtgacggaggagctgaagcacatgctgtctcctcagagagcaccagagagactaatgcagctggccgacggcaacctgggaagcctggtcactgagatggacgaactgcttaaccgggcaactaaagtgtctgctgatggggagcagacggcagcggatgcagagagaagccgtaaagctgcagaagagctggaactgtacgtcaggaacacactgctggctgctgaagcactgcgtgacaaagcacgtgatctgaacgcgactttgggacttcgggatgggactttagagaaaagcgtgaacgagatgaatgacgagatccaggaaatgctcgcagagctccgcaaacgccagctcaccggaaagaaacacatggctgatgaagagctcggggctgcggaggctctcttgtcccgcgtgaagcgcttgtttggagacccacagcaggccacagcggacctgaagcaggaggtgggacagaagatgagcgaccacaagctgaagctgcaggaggcgcaggagctgctggaggaggcgctcacacacaccagacaggctgaagacctgactctcaacaaccagctcaaactgcagcatctgcagggaaagagggatgcggccgttaaacagaaagaacaaacagagggggtcttattggagggagagaagagtctggacgaagccaatgctctctccgacgccatcacccgcggcaaagaggagctggaggagatggcgggagagctggggcctctgcgggatcagctgcgggataaagagtctgatctgagtcgcggtctggatgagtctgtcccggagctgctgctaagagcggaggatcacgccgcacagctcaaccactccgccgccatactcgacagtattctggctgaggcaaagaatctgtccttcaacgccacggctgctttcagcgcatacaccaacatcaagaactacatcgaggaggccgatagagtggccaaagatgcaaagaaaaccagcgctgaggctctacagctggcctcgggacccaaaggctctctgaaagatcaggctaaaggttcgctccagaagagccacgatctgctgaatgaggctaaagacctgcagaaggacgtgacagaaaatggagagaatctgggagcccttcagctcagactgaaaggagccaacaagcagaacaaagacctacagaaacgcctgaacgacaccgtggagaaactcaactccatccctgacgatatggcgctgaagatccaggctactaagctgaaggcggccgaagccaacgacacggcgctggacgtcctgaatcggctgaaggacataaacctcaacctgatgggcttgaagaaaaactacgacaagctggaggacgacgtcaggaaaaccaacaacctgatcaaagaccccgagaaaaacattcatgcagcaggcgccaaagtgaaggatctggagaatgaagccgacaggctgatggagaagctgaagcccattcaggagctgcaggacaacctgaagaaaaacatctcgcagattaaagaactcatcaaccaggcacgcaagcaggccaactctattaaggtgtctgtgttgtccggtggagactgcatccgcacctaccggccagagatcaagaagggccgctacaacaccatcatcctccacctcaagaccctcgctgccgataacctgctcttctacctgggatccgccaaatacgtggatttcttggccatcgagatgcgtaaggggaaggtgaacttcctgtgggatgtggggtcaggggtcggccgagtggaatatccagatctgatcatcaacgatggaaactggcaccgaatcgaagcctcacgtattggtctgaacggcagtatctcagtccgtgcgctcgagggacccaaaacaggcatcttaccttcactcagaagtgcaaaagcaccggagacctacactgtgctggacgtggaccagaacgcttacctgtttgtcggaggcatgctgggatcagtgaagaaagcagatgctgtgaaaaccaccacattctcagggtgtatgggtgagacgtctctggatggaaagcccatcggcctctggaactacagagagagagacggggactgtgctggatgtgttgtcagtcctcagcctgcagacactgaaggaacggttcagtttgatggagatggttatgcgtccgtcagtcgccccaccaggtggaacccaaatgtctccactgtcatgttcaagtttcgcaccttctccacacactccgtcatcatgtacttcgccaccaaagacatgaaagacttcatgagtgctgaactgagtgatggcagggtgaaagtgagttatgatctgggctctggcaccggctccatcatcagtgataaacgctataatgacggcaagtggaagtccttcaccatgtcccgcatgaagaaagaagccaccatcaccatcgtcaacatggacactaatgagaatgagcggctgctgatggcgtcaccagggggcgctacaggccttaacctgcgagacaacgagcgcatatttttcggaggattgcccaaagctggaaaatacaggtcagaggtggttttgaagaggttttctggctgtatgaaggatatcgaggtctcccgaacgccatataatctgctgagcagtccagattacacaggactgaccaaaggctgctccattgagaatctgcacacagtgagtttcccacgggccgggttcatggagctcaatccggtttcgtttgccatgggctcagagatctcgctctccttcagcaccaggagtgaagacgggctgatactgttcggcagaggaggactgacctccatgacggagctgacacagggactcacgccagtgcacatcccacgccgctcacgcagacagaccggagagcccttcctctctgtgcagctcagtaaaggggcactggaggtgctggtgttcaccggctccagaaaccctcgcagagttttccgaaaaccagatcagggaatcctgcatgatggccgagagcactcactgcgcatccagaggctcccgggagcatccttcactgtgcaggtggacgaggagccgctggtgcagcagcccttacccaacgacctcccactcaacatccaccgcctcttcatcggaggaatcccgcctgatgtacaaaccggcgtccacaggcccagcgtgccgttcgagggctgcatctggaacctcctgatcaacactgtcccggtggacttctcccagcctgtggcctttgagaatgcagagattggccactgccctgacctggccccggccccagctccgcctcttgaggaggtggaaaaggaagaggctccgcctacaccagcagaagcagttcgtcctccaccagctccagcctccactgccacaccccttgccgaagccaagcccatcacagacacacccaccagtgtcaaagagcccgcggtgtcgtgcgcagcagaggcggagcctgcagtgctggggctggccaaacagttcggtctgtccagaaacagtcacatgagctttgagtttgatgacagaaaggtgatgaacaggttggtcattgagttcgagatgcgcacagagacagactctggtctggttttctacatggccagaataaaccacgcagacttcgccaccatccagctgaaggagggaatggctcacctaagcttcgacctgggcagcgggaacacctccgtcagcgtgccgcgcatcatcaacgacgggcagtgacacaagatccgtgtgatgagagacaaacagagaggagtcctcacgatagacggccgctacagcaaacacaccaccagcccgaagaaagcagaaatcctggatgtggtgggaatgctgtatgttggtggtttaccgctcaactacaccaccaaacgcatcggccctgtgctgtacagtattgacgcttgcattaggaacttcaagatgatgaatctgcctttggatatggagaatcccacctccagctaccgcgtgggctcctgctttgccaacccagaaaagggaaactatttcaatggcacaggctatgccaaagctgtggaggcatatcgagtgggtaatgacgtgtcagtggatctggagttcaggacggctcagagctctgctgtcctggtgggcgtcagcagccaaaagatggacgggctgggcatcgaactcgtaaatggaaagctgttcttccatgctgataacggagtgggccgcttcacagccgtgcatgagccgaggcaggaggcggggctttgtgatgggcagtggcacacagtctccgcccacaagctgaagcaccgcctggagctgacggtggacggacagaagtctgaggcggcgagtccagacatccgctcgccctccgcagacaccaatgacccactgtatgtgggcggataccccgagggtgggaagcagttcgggctcaccataaacaccccgtttaaaggctgcatgaggaacgtgaagatcagtaaagctgggaaaactctgcaggtccagatgaacaaactgctggagctcagaggagttcagccgctcagctgccctgctgcttaa